(19) United States
(12) Patent Application Publication
(10) Pub. No.: US 2005/0142555 A1
(43) Pub. Date: Jun. 30, 2005

(54) Novel dendritic cell-specific polynucleotides and microarray comprising the same

(76) Inventors: Jung-Hoon Anh, Daejon Metropolitan City (KR); Yoon Lee, Daejeon Metropolitan City (KR); Choon-ju Jeon, Daejeon Metropolitan City (KR); Byung-Hak Lee, Daejeon Metropolitan City (KR); Kang-Duk Choi, Daejeon Metropolitan City (KR); Yong-Soo Bae, Daejeon Metropolitan City (KR)

Correspondence Address: NIXON & VANDERHYE, PC, 1100 N GLEBE ROAD, 8TH FLOOR, ARLINGTON, VA, 22201-4714, US

(21) Appl. No.: 10/509,621

(22) PCT Filed: Mar. 28, 2003
(86) PCT No.: PCT/KR03/00631

(30) Foreign Application Priority Data
Mar. 29, 2002 (KR) 10-2002-17470

(51) Int. Cl. C12Q 1/68; C07H 21/04; C12M 1/34

(52) U.S. Cl. 435/6; 435/287.2; 536/23.1

(57) ABSTRACT

The present invention relates to dentritic cell-specific polynucleotides and microarrays, particularly to novel polynucleotides highly expressed in dendritic cells, certain dendritic cell subsets and matured dendritic cells and microarrays comprising thereof. With a marrcoarray of this invention, dendritic cells, certain dendritic cells subsets and matured dendritic cells can be detected rapidly and accurately.

### FIELD OF THE INVENTION

The present invention relates to dendritic cell-specific polynucleotides and microarrays, particularly to novel polynucleotides highly expressed in dendritic cells, specific dendritic cell subsets and matured dendritic cells and microarrays comprising them.

### DESCRIPTION OF THE RELATED ART

Dendritic cells (DCs) are specialized to modulate T cell immunity either by priming or tolerating antigen (Ag)-specific T cells, depending on the exact physiological conditions, such as the nature and amount of Ag and the presence of DC-maturating stress signals (1-4). While comprising less than 1% of the total mononuclear cells in mouse spleen and human peripheral blood, DCs are present ubiquitously in all tissues, even in the human CNS (5). Unlike other immune cells, DCs arise, upon different signals, from many different progenitor cells of myeloid or lymphoid origin (6-8). The heterogeneity of the DC population is well demonstrated by the multiple DC subsets in human blood and mouse spleens. Although the ontogeny of each type of DC still remains unclear, the presence of multiple distinct DC lineages in both human and mouse has raised the possibility that distinct DC subsets might have unique functions recruiting distinct types of immune responses (9-12). Intriguingly, it has been noted that even for a given type of DC, there is considerable plasticity in DC functions depending on the maturation stage and the duration of Ag-exposure, resulting in different outcomes of DC-mediated immune triggering (13-17).

Due to their pivotal role in immune induction and tolerance, DCs have been explored for their use in the control of malignant cancers and autoimmune diseases in mouse models (18,19). However, considering the heterogeneity of naturally occurring DCs, the current DC study in association with human immunotherapy might have been skewed in monocyte-derived dendritic cells (MoDCs). Indeed, many clinical trials using MoDCs are being undertaken to elicit tumor-specific immunities (20,21). Increasing pressure from translational research, however, necessitates the study of other human DCs which might be useful to control harmful immune responses such as autoimmunity and graft rejection.

In the last few years, advances in methodology have enabled us to access various human DCs of high purity and good quality (22-25). In order to have better insight into the unique capacities of distinct DC subsets, attempts have been made to disclose DC-associated genes and their expression patterns. Several independent approaches have been made to reveal the genes highly expressed in MoDCs, by employing sequential analysis of gene expression (SAGE) or cDNA microarray system (26,27). The expression profiles from these studies are generally in good agreement with each other and sufficient to arrive at a consensus about genes which are highly expressed in MoDCs. However, it remains to be addressed whether these genes are also prominent in other types of DCs. Performing differential plaque lifting hybridization and differential display RT-PCR, the present inventors identified DC-specific genes from low-density blood DCs (30, 31). The inventors pooled out the “DC-associated genes” from three different DC subsets, namely, CD11c⁻ DCs isolated from peripheral blood (23), CD1a⁺ DCs and CD14⁺ DCs (24,25,32 and 33) generated from hematopoietic progenitor cells.

### DETAILED DESCRIPTION OF THIS INVETNION

The present inventors have made intensive research to identify the nucleotide sequences specific to dendritic cells (DCs), certain DC subsets and/or matured DCs and as a result, found a number of DC-specific nucleotide sequences including novel nucleotide sequences, thereby accomplishing the present invention.

Accordingly, it is an object of this invention to provide a novel dendritic cell-specific polynucleotide.

It is another object of this invention to provide a polypeptide encoded by the dendritic cell-specific nucleotide sequence.

It is still another object of this invention to provide a method for detecting a dendritic cell.

It is another object of this invention to provide a method for identifying DC subsets (a lymphoid CD11c− dendritic cell, a myeloid monocyte-derived dendritic cell, a myeloid CD1a⁺ dendritic cell and a myeloid CD14⁺ dendritic cell).

It is another object of this invention to provide a method for identifying a maturation stage of a dendritic cell subset.

It is further object of this invention to provide a microarray for detecting a dendritic cell.

It is still further object of this invention to provide a microarray for identifying a dendritic cell subset.

It is another object of this invention to provide a microarray for identifying a maturation stage of a dendritic cell subset.

In one aspect of this invention, there is provided a dendritic cell-specific polynucleotide comprising a nucleotide sequence of SEQ ID NO:1.

In another aspect of this invention, there is provided a dendritic cell-specific polynucleotide comprising a nucleotide sequence of SEQ ID NO:2.

In still another aspect of this invention, there is provided a dendritic cell-specific polynucleotide comprising a nucleotide sequence of SEQ ID NO:3.

In another aspect of this invention, there is provided a dendritic cell-specific polynucleotide comprising a nucleotide sequence of SEQ ID NO:4.

In another aspect of this invention, there is provided a dendritic cell-specific polynucleotide comprising a nucleotide sequence of SEQ ID NO:5.

In another aspect of this invention, there is provided a dendritic cell-specific polynucleotide comprising a nucleotide sequence of SEQ ID NO:6.

It is in dendritic cells that these polynucleotides of this invention are expressed specifically. In general, they show high expression patterns, and particularly, some of them are highly expressed only in certain dendritic cell subsets and others only in matured dendritic cells. Thus, these polynucleotides of this invention can be usefully applied to detection of dendritic cells and identification of dendritic cell subsets and/or matured dendritic cells.

In another aspect of this invention, there is provided a dendritic cell-specific polypeptide encoded by a nucleotide sequence of SEQ ID NO:1.

In still another aspect of this invention, there is provided a dendritic cell-specific polypeptide encoded by a nucleotide sequence of SEQ ID NO:2.

In another aspect of this invention, there is provided a dendritic cell-specific polypeptide encoded by a nucleotide sequence of SEQ ID NO:3.

In another aspect of this invention, there is provided a dendritic cell-specific polypeptide encoded by a nucleotide sequence of SEQ ID NO:4.

In still another aspect of this invention, there is provided a dendritic cell-specific polypeptide encoded by a nucleotide sequence of SEQ ID NO:5.

In another aspect of this invention, there is provided a dendritic cell-specific polypeptide encoded by a nucleotide sequence of SEQ ID NO:6.

In another aspect of this invention, there is provided a method for detecting a dendritic cell comprising the steps of: (a) hybridizing a DNA obtained from a cell or its fragment with a dendritic cell-specific nucleotide sequence; and (b) verifying the occurrence of the hybridization;
wherein said dendritic cell-specific nucleotide sequence is selected from the group consisting of myosin phosphatase, target subunit 1 (MYPT1) gene, CD20-like precursor gene, Ig superfamily protein (Z39IG) gene, glycoprotein nmb (GPNMB) gene, 5-lipoxygenase activating protein (FLAP) gene, dihydropyrimidinase related protein-2 gene, cystatin A (CSTA) gene, Immunoglobulin transcription factor 2 (IFT2) gene, transforming growth factor beta-induced 68 kD (TGFBI) gene, myeloid DAP12-associating lectin (MDL-1) gene, B cell linker protein (BLNK) gene, Activated RNA polymerase II transcription cofactor 4 (PC4), enolase 1 alpha (ENO1) gene, 90 kDa heat shock protein (hsp90) gene, accessory proteins BAP31/BAP29 gene, isocitrate dehydrogenase 3 (NAD⁺) alpha (IDH3A) gene, microsomal glutathione S-transferase 2 (MGST2) gene, GABA(A) receptor-associated protein (GABARAP) gene, nicastrin gene, purinergic receptor (family A group 5) gene, Rho GDP dissociation inhibitor beta (ARHGDIB) gene, MAD homolog 2 (MADH2) gene, MLN51 gene, interferon regulatory factor 4 (IRF4) gene, the fragments of these genes, a polynucleotide of SEQ ID NO:1 or its fragment, a polynucleotide of SEQ ID NO:2 or its fragment, a polynucleotide of SEQ ID NO:3 or its fragment, a polynucleotide of SEQ ID NO:4 or its fragment, a polynucleotide of SEQ ID NO:5 or its fragment, a polynucleotide of SEQ ID NO:6 or its fragment and the combination thereof.

In still another aspect of this invention, there is provided a method for identifying a lymphoid CD11c⁻ dendritic cell comprising the steps of: (a) hybridizing a DNA obtained from a cell or its fragment with a CD11c⁻ dendritic cell-specific nucleotide sequence; and (b) verifying the occurrence of the hybridization;
wherein said lymphoid CD11c⁻ dendritic cell-specific nucleotide sequence is selected from the group consisting of 5-lipoxygenase activating protein gene or its fragment, dihydropyrimidinase related protein-2 gene or its fragment, interferon regulatory factor 4 gene or its fragment and the combination thereof.

In another aspect of this invention, there is provided a method for identifying a myeloid monocyte-derived dendritic cell comprising the steps of: (a) hybridizing a DNA obtained from a cell or its fragment with a myeloid monocyte-derived dendritic cell-specific nucleotide sequence; and (b) verifying the occurrence of the hybridization;
wherein said myeloid monocyte-derived dendritic cell-specific nucleotide sequence is selected from the group consisting of thymus and activation-regulated chemokine (TARC) gene or its fragment, dihydropyrimidinase related protein-2 gene or its fragment, lysosomal acid lipase gene or its fragment, calmodulin gene or its fragment, interferon regulatory factor 4 gene or its fragment, DC-Lamp gene or its fragment and the combination thereof.

In another aspect of this invention, there is provided a method for identifying a myeloid CD1a⁺ dendritic cell comprising the steps of: (a) hybridizing a DNA obtained from a cell or its fragment with a myeloid CD1a⁺ dendritic cell-specific nucleotide sequence; and (b) verifying the occurrence of the hybridization;
wherein said myeloid CD1a⁺ dendritic cell-specific nucleotide sequence is selected from the group consisting of a polynucleotide of SEQ ID NO:2 or its fragment, a polynucleotide of SEQ ID NO:3 or its fragment, a polynucleotide of SEQ ID NO:5 or its fragment, S100 calcium-binding protein beta (S100B) gene or its fragment, matrix metalloproteinase 12 (MMP12) gene or its fragment, thymus and activation-regulated chemokine (TARC) gene or its fragment, CD1B antigen (CD1B) gene or its fragment, CD20-like precursor gene or its fragment, MHC class II HLA-DQ-alpha chain gene or its fragment, osteopontin (Eta-1) gene or its fragment, 5-lipoxygenase activating protein gene or its fragment, monocyte chemotactic proteins 4 (MCP4) gene or its fragment, lysosomal acid lipase gene or its fragment, cystatin A gene or its fragment, annexin A2 (ANXA2) gene or its fragment, vesicle-associated membrane protein 8 (VAMP8) gene or its fragment, MHC class II HLA-DM-alpha chain (MHC DM-alpha) gene or its fragment, DORA protein gene or its fragment, DC-Lamp gene or its fragment, Mannose receptor (CD206) gene or its fragment, Langerin (CD207) gene or its fragment and the combination thereof.

In still another aspect of this invention, there is provided a method for identifying a myeloid CD14⁺ dendritic cell comprising the steps of: (a) hybridizing a DNA obtained from a cell or its fragment with a myeloid CD14⁺ dendritic cell-specific nucleotide sequence; and (b) verifying the occurrence of the hybridization;
wherein said myeloid CD14⁺ dendritic cell-specific nucleotide sequence is selected from the group consisting of a polynucleotide of SEQ ID NO:2 or its fragment, S100 calcium-binding protein beta gene or its fragment, myosin phosphatase target subunit 1 gene or its fragment, CD20-like precursor gene or its fragment, Ig superfamily protein gene or its fragment, glycoprotein nmb gene or its fragment, osteopontin gene or its fragment, 5-lipoxygenase activating protein gene or its fragment, mannose receptor C type 1 (MRC1) gene or its fragment, monocyte chemotactic proteins 4 gene or its fragment, RNase A family 1 (RNas1) gene or its fragment, lysosomal acid lipase gene or its fragment, cystatin A gene or its fragment, monocyte chemotactic proteins 1 (MCP 1) gene or its fragment, transforming growth factor beta-induced 68 kD gene or its fragment, ferritin light polypeptide gene or its fragment, vesicle-associated membrane protein 8 gene or its fragment, Mannose receptor (CD206) gene or its fragment and the combination thereof.

In another aspect of this invention, there is provided a method for identifying a maturation stage of a lymphoid CD11c⁻ dendritic cell comprising the steps of: (a) hybridizing a DNA obtained from a cell or its fragment with an interferon regulatory factor 4 gene or its fragment; and (b) verifying the occurrence of the hybridization.

In still another aspect of this invention, there is provided a method for identifying a maturation stage of a myeloid monocyte-derived dendritic cell comprising the steps of: (a) hybridizing a DNA obtained from a cell or its fragment with a nucleotide sequence; and (b) verifying the occurrence of the hybridization;
wherein said nucleotide sequence is selected from the group consisting of thymus and activation-regulated chemokine gene or its fragment, dihydropyrimidinase related protein-2 gene or its fragment, interferon regulatory factor 4 gene or its fragment, DC-Lamp gene or its fragment and the combination thereof.

In another aspect of this invention, there is provided a method for identifying a maturation stage of a myeloid CD1a⁺ dendritic cell comprising the steps of: (a) hybridizing a DNA obtained from a cell or its fragment with a nucleotide sequence; and (b) verifying the occurrence of the hybridization;
wherein said nucleotide sequence is selected from the group consisting of a polynucleotide of SEQ ID NO:2 or its fragment, a polynucleotide of SEQ ID NO:3 or its fragment, a polynucleotide of SEQ ID NO:5 or its fragment, S100 calcium-binding protein beta gene or its fragment, matrix metalloproteinase 12 gene or its fragment, thymus and activation-regulated chemokine gene or its fragment, CD1B antigen gene or its fragment, CD20-like precursor gene or its fragment, MHC class II HLA-DQ-alpha chain gene or its fragment, osteopontin gene or its fragment, monocyte chemotactic proteins 4 gene or its fragment, lysosomal acid lipase gene or its fragment, cystatin A gene or its fragment, transforming growth factor beta-induced 68 kD gene or its fragment, annexin A2 gene or its fragment, vesicle-associated membrane protein 8 gene or its fragment, DORA protein gene or its fragment, DC-Lamp gene or its fragment, Langerin (CD207) gene or its fragment and the combination thereof.

In another aspect of this invention, there is provided a method for identifying a maturation stage of a myeloid CD14⁺ dendritic cell comprising the steps of: (a) hybridizing a DNA obtained from a cell or its fragment with a nucleotide sequence; and (b) verifying the occurrence of the hybridization;
wherein said nucleotide sequence is selected from the group consisting of a polynucleotide of SEQ ID NO:2 or its fragment, S100 calcium-binding protein beta gene or its fragment, CD20-like precursor gene or its fragment, Ig superfamily protein gene or its fragment, glycoprotein nmb gene or its fragment, osteopontin gene or its fragment, 5-lipoxygenase activating protein gene or its fragment, mannose receptor C type 1 gene or its fragment, monocyte chemotactic proteins 4 gene or its fragment, RNase A family 1 gene or its fragment, lysosomal acid lipase gene or its fragment, cystatin A gene or its fragment, monocyte chemotactic proteins 1 gene or its fragment, transforming growth factor beta-induced 68 kD gene or its fragment, ferritin light polypeptide gene or its fragment, vesicle-associated membrane protein 8 gene or its fragment, Mannose receptor (CD206) gene or its fragment and the combination thereof.

According to a method of this invention, it is preferred that the preparation of a DNA from cell to be analyzed is performed by reverse-transcripting mRNA isolated from the cell to obtain cDNA. In a specific example, RT-PCR (reverse transcriptase-PCR) is carried out to prepare cDNA.

The DNA (e.g. cDNA) prepared thus is preferably labeled. For labeling, materials detectable by spectroscopic measurement, photochemical measurement, biochemical measurement, bioelectronic measurement, immunochemical measurement, electronic measurement, chemical measurement are used. For instance, the labels include, but not limited to, radioisotopes such as P³² and S³⁵, chemilluminescent compounds, labeled binding proteins, heavy metal atoms, spectroscopic markers such as fluorescence markers and dyes, and magnetic labels. The dyes, for example, include, but not limited to, quinoline dye, triarylmethane dye, phthalein, azo dye and cyanine dye. The fluorescence makers include, but not limited to, fluorescein, phycoerythrin, rhodamine, lissamine, Cy3 and Cy5 (Pharmacia). Labeling is performed according to various methods known in the art, such as nick translation, random priming (Multiprime DNA labeling systems booklet, “Amersham” (1989)) and kination (Maxam & Gilbert, Methods in Enzymology, 65: 499 (1986)).

According to a method of the present invention, the hybridization of a DNA obtained from a cell with a dendritic cell-specific nucleotide sequence is carried out with referring to the procedures described in Southern, E. J. Mol. Biol. 98: 503 (1975) in the hybridization conditions optimized through modifying several factors (salt concentration, temperature, reaction time and probe concentration) (Molecular Cloning, A Laboratory Manual, 2nd ed., 9.52-9.55 (1989)).

In a preferable embodiment of this invention, the differential hybridization is adopted for hybridization. Differential hybridization is generally performed in such a manner that DNAs prepared from two sources are labeled with different labels (e.g. Cy3 and Cy5) respectively and DNAs labeled are hybridized with the nucleotide sequences described above to detect and analyze two signals.

According to a method of the present invention, the occurrence of hybridization is verified with various methods known in the art, particularly, depending on the types of labels used. For example, fluorescence microscope, preferably, confocal fluorescence microscope is used for fluorescence labels, and the intensity of the signal detected with such instruments increases proportionally to the extent of hybridization. Fluorescence microscopes, in general, are equipped with a scanning device which builds up a quantitative two dimensional image of hybridization intensity. The scanned image allows for the identification of a dendritic cell, a dendritic cell subset and/or a maturation stage of a dendritic cell.

The nucleotide sequences (polynucleotides or oligonucleotides) used in the present method are high-expressed specifically in a dendritic cell (at the stage of transcription and/or translation), which have been firstly revealed by the present inventors.

The method of the present invention detects successfully dendritic cells in cell samples derived from various biological sources (tissue, blood, etc.).

In a method of the present invention for identifying a maturation stage of a lymphoid CD11c⁻ dendritic cell, the decrease of hybridization signal measured with an interferon regulatory factor 4 gene or its fragment as probe indicates a lymphoid CD11c⁻ dendritic cell matured unlike other method for identifying a maturation stage of a dendritic cell.

In another aspect of this invention, there is provided a microarray for detecting a dendritic cell comprising a dendritic cell-specific nucleotide sequence immobilized on a solid surface;
wherein said dendritic cell-specific nucleotide sequence is selected from the group consisting of myosin phosphatase target subunit 1 (MYPT1) gene, CD20-like precursor gene, Ig superfamily protein (Z39IG) gene, glycoprotein nmb (GPNMB) gene, 5-lipoxygenase activating protein gene, dihydropyrimidinase related protein-2 gene, cystatin A (CSTA) gene, Immunoglobulin transcription factor 2 (IFT2) gene, transforming growth factor beta-induced 68 Kd (TGFBI) gene, myeloid DAP12-associating lectin (MDL-1) gene, B cell linker protein (BLNK) gene, activated RNA polymerase II transcription cofactor 4 (PC4) gene, enolase 1 alpha (ENO1) gene, 90 kDa heat shock protein (hsp90) gene, accessory proteins BAP31/BAP29 gene, isocitrate dehydrogenase 3 (NAD⁺) alpha (IDH3A) gene, microsomal glutathione S-transferase 2 (MSGT2) gene, GABA(A) receptor-associated protein (GABARAP) gene, nicastrin gene, purinergic receptor (family A group 5) gene, Rho GDP dissociation inhibitor beta (ARHGDIB) gene, MAD homolog 2 (MADH2) gene, MLN51 gene, interferon regulatory factor 4 (IRF4) gene, the fragments of these genes, a polynucleotide of SEQ ID NO:1 or its fragment, a polynucleotide of SEQ ID NO:2 or its fragment, a polynucleotide of SEQ ID NO:3 or its fragment, a polynucleotide of SEQ ID NO:4 or its fragment, a polynucleotide of SEQ ID NO:5 or its fragment, a polynucleotide of SEQ ID NO:6 or its fragment and the combination thereof.

In still another aspect of this invention, there is provided a microarray for identifying a lymphoid CD11c⁻ dendritic cell comprising a lymphoid CD11c⁻ dendritic cell-specific nucleotide sequence immobilized on a solid surface;
wherein said lymphoid CD11c⁻ dendritic cell-specific nucleotide sequence is selected from the group consisting of 5-lipoxygenase activating protein gene or its fragment, dihydropyrimidinase related protein-2 gene or its fragment, interferon regulatory factor 4 gene or its fragment and the combination thereof.

In another aspect of this invention, there is provided a microarray for identifying a myeloid monocyte-derived dendritic cell comprising a myeloid monocyte-derived dendritic cell-specific nucleotide sequence immobilized on a solid surface;
wherein said myeloid monocyte-derived dendritic cell-specific nucleotide sequence is selected from the group consisting of thymus and activation-regulated chemokine (TARC) gene or its fragment, dihydropyrimidinase related protein-2 gene or its fragment, lysosomal acid lipase or its fragment, calmodulin gene or its fragment, interferon regulatory factor 4 gene or its fragment, DC-Lamp gene or its fragment and the combination thereof.

In another aspect of this invention, there is provided a microarray for identifying a myeloid CD1a⁺ dendritic cell comprising a myeloid CD1a⁺ dendritic cell-specific nucleotide sequence immobilized on a solid surface;
wherein said myeloid CD1a⁺ dendritic cell-specific nucleotide sequence is selected from the group consisting of a polynucleotide of SEQ ID NO:2 or its fragment, a polynucleotide of SEQ ID NO:3 or its fragment, a polynucleotide of SEQ ID NO:5 or its fragment, S100 calcium-binding protein beta (S100B) gene or its fragment, matrix metalloproteinase 12 (MMP 12) gene or its fragment, thymus and activation-regulated chemokine gene or its fragment, CD1B antigen (CD1B) gene or its fragment, CD20-like precursor gene or its fragment, MHC class II HLA-DQ-alpha chain (MHC DQ-alpha) gene or its fragment, osteopontin (Eta-1) gene or its fragment, 5-lipoxygenase activating protein gene or its fragment, monocyte chemotactic proteins 4 (MCP4) gene or its fragment, lysosomal acid lipase gene or its fragment, cystatin A gene or its fragment, annexin A2 (ANXA2) gene or its fragment, vesicle-associated membrane protein 8 (VAMP8) gene or its fragment, MHC class II HLA-DM-alpha chain gene (MHC DM-alpha) or its fragment, DORA protein gene or its fragment, DC-Lamp gene or its fragment, Mannose receptor (CD206) gene or its fragment, Langerin (CD207) gene or its fragment and the combination thereof.

In another aspect of this invention, there is provided a microarray for identifying a myeloid CD14⁺ dendritic cell comprising a myeloid CD14⁺ dendritic cell-specific nucleotide sequence immobilized on a solid surface;
wherein said myeloid CD14⁺ dendritic cell-specific nucleotide sequence is selected from the group consisting of a polynucleotide of SEQ ID NO:2 or its fragment, S100 calcium-binding protein beta gene or its fragment, myosin phosphatase target subunit 1 gene or its fragment, CD20-like precursor gene or its fragment, Ig superfamily protein gene or its fragment, glycoprotein nmb gene or its fragment, osteopontin gene or its fragment, 5-lipoxygenase activating protein gene or its fragment, mannose receptor C type 1 (MRC1) gene or its fragment, monocyte chemotactic proteins 4 gene or its fragment, RNase A family 1 (RNas 1) gene or its fragment, lysosomal acid lipase gene or its fragment, cystatin A gene or its fragment, monocyte chemotactic proteins 1 (MCP 1) gene or its fragment, transforming growth factor beta-induced 68 kD gene or its fragment, ferritin light polypeptide gene or its fragment, vesicle-associated membrane protein 8 gene or its fragment, Mannose receptor (CD206) gene or its fragment and the combination thereof.

In still another aspect of this invention, there is provided a microarray for identifying a maturation stage of a lymphoid CD11c⁻ dendritic cell comprising an interferon regulatory factor 4 gene or its fragment immobilized on a solid surface.

In another aspect of this invention, there is provided a microarray for identifying a maturation stage of a myeloid monocyte-derived dendritic cell comprising a nucleotide sequence immobilized on a solid surface;
wherein said nucleotide sequence is selected from the group consisting of thymus and activation-regulated chemokine gene or its fragment, dihydropyrimidinase related protein-2 gene or its fragment, interferon regulatory factor 4 gene or its fragment, DC-Lamp gene or its fragment and the combination thereof.

In another aspect of this invention, there is provided a microarray for identifying a maturation stage of a myeloid CD1a⁺ dendritic cell comprising a nucleotide sequence immobilized on a solid surface;
wherein said nucleotide sequence is selected from the group consisting of a polynucleotide of SEQ ID NO:2 or its fragment, a polynucleotide of SEQ ID NO:3 or its fragment, a polynucleotide of SEQ ID NO:5 or its fragment, S100 calcium-binding protein beta gene or its fragment, matrix metalloproteinase 12 gene or its fragment, thymus and activation-regulated chemokine gene or its fragment, CD1B antigen gene or its fragment, CD20-like precursor gene or its fragment, MHC class II HLA-DQ-alpha chain gene or its fragment, osteopontin gene or its fragment, monocyte chemotactic proteins 4 gene or its fragment, lysosomal acid lipase gene or its fragment, cystatin A gene or its fragment, transforming growth factor beta-induced 68 kD gene or its fragment, annexin A2 gene or its fragment, vesicle-associated membrane protein 8 gene or its fragment, DORA protein gene or its fragment, DC-Lamp gene or its fragment, Langerin (CD207) gene or its fragment and the combination thereof.

In another aspect of this invention, there is provided a microarray for identifying a maturation stage of a myeloid CD14⁺ dendritic cell comprising a nucleotide sequence immobilized on a solid surface;
wherein said nucleotide sequence is selected from the group consisting of a polynucleotide of SEQ ID NO:2 or its fragment, S100 calcium-binding protein beta gene or its fragment, CD20-like precursor gene or its fragment, Ig superfamily protein gene or its fragment, glycoprotein nmb gene or its fragment, osteopontin gene or its fragment, 5-lipoxygenase activating protein gene or its fragment, mannose receptor C type 1 gene or its fragment, monocyte chemotactic proteins 4 gene or its fragment, RNase A family 1 gene or its fragment, lysosomal acid lipase gene or its fragment, cystatin A gene or its fragment, monocyte chemotactic proteins 1 gene or its fragment, transforming growth factor beta-induced 68 kD gene or its fragment, ferritin light polypeptide gene or its fragment, vesicle-associated membrane protein 8 gene or its fragment, Mannose receptor (CD206) gene or its fragment and the combination thereof.

In a microarry of this invention, the genes or their fragments are used as hybridizable array elements and immobilized on a substrate. A preferable substrate includes suitable solid or semi-solid supporters, such as membrane, filter, chip, slide, wafer, fiber, magnetic or nonmagnetic bead, gel, tubing, plate, macromolecule, microparticle and capillary tube. The hybridizable array elements are arranged and immobilized on the substrate. Such immobilization occurs through chemical binding or covalent binding such as UV. In an embodiment of this invention, the hybridizable array elements are bound to a glass surface modified to contain epoxi compound or aldehyde group or to a polylysin-coated surface. Further, the hybridizable array elements are bound to a substrate through linkers (e.g. ethylene glycol oligomer and diamine).

DNAs to be examined with a microarry of this invention are labeled as describe above, and hybridized with array elements on microarray. Various hybridization conditions are applicable as mentioned previously, and are exemplified in the Examples below.

For the detection and analysis of the extent of hybridization, various methods are available depending on labels used, and are exemplified in the Examples below.

Also, in a preferable embodiment, a microarray of this invention includes spike genes. Spike genes play a role in the correction of the signal difference occurring during hybridization on microarray with DNA or RNA fluorescence labeling.

With a mircoarray of this invention, dendritic cells, specific dendritic cell subsets and a maturation stage of dendritic cells can be detected.

The present invention will now be described in further detail by examples. It would be obvious to those skilled in the art that these examples are intended to be more concretely illustrative and the scope of the present invention as set forth in the appended claims is not limited to or by the examples.

### BRIEF DESCRIPTION OF THE DRAWINGS

FIG. 1a schematically shows the procedures for generating and isolating each DC subset. Lin⁻ means TCR⁻CD14⁻CD16⁻CD19⁻CD56⁻.

FIG. 1b is FACS graphs and photographs showing the surface phenotype of each DC subset in immature and mature stages. Photographs of DC subsets on the right hand side were taken on day 18, day 18, day 5 and day 9 for CD1a⁺ DC, CD14⁺ DC, CD11c⁻ DC and MoDC, respectively.

FIG. 2 is a brief scheme showing entire strategy for identification and characterization of DC-associated genes. DC denotes an equal mixture of three different DC subsets. BMT denotes the mixture of B cells, monocytes and T cells in equal amounts.

FIG. 3 represents the results of a semi-quantitative RT-PCR analysis showing the expression profile of DC-associated genes in each DC subset. In Figure, the results of RT-PCR are summarized with differential marking: (+++) for higher expression and (++) for lower expression detectable after 25 cycles of PCR, (+) and (+/−) for expression detectable only after 30 cycles of PCR and marginally detectable even after 30 cycles of PCR, respectively. CD19, CD14, and CD28 were used as control genes for B cells, monocytes, and T cells, respectively. Genes not detected by differential screening were indicated by asterisk (*). ND denotes ‘not determined’ in this study.

### EXAMPLES

Methods

Cell and RNA Preparations

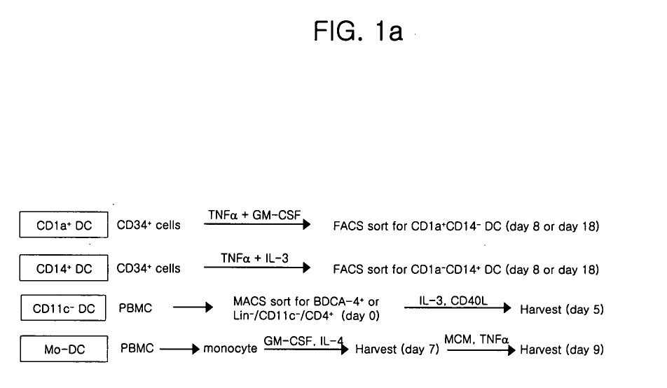

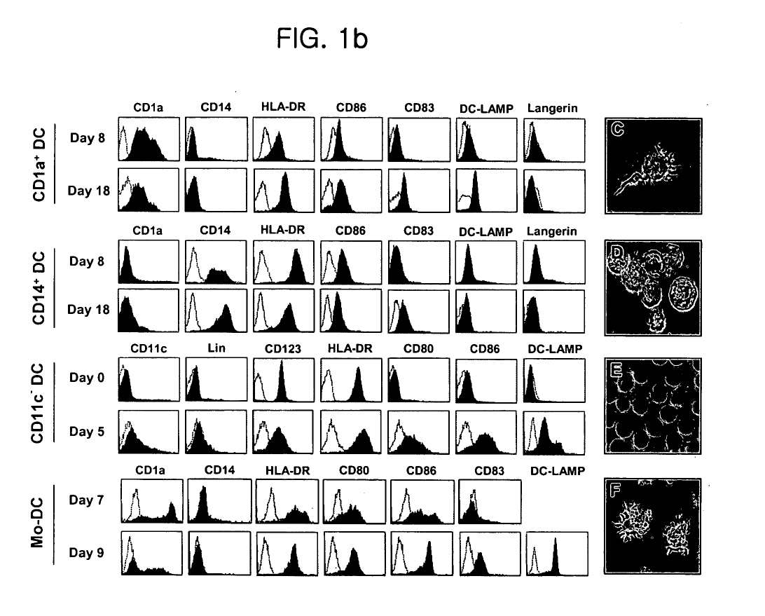

Generation or isolation procedures of each DC subset were summarized in FIG. 1a. CD1a⁺ DCs or CD14⁺ DCs were generated from CD34⁺ progenitor cells isolated from umbilical cord blood (supplied from the College of Medicine, Chungnam National University and the Motae Obstetrician's Offices, Taejon, Korea). Mononuclear cells (PBMC) from umbilical cord blood were obtained by a standard Ficoll density gradient method (d 1.077 g/ml). CD34⁺ hematopoietic progenitor cells were isolated from the PBMC using a MACS separation kit (Miltenyi Biotec). CD34⁺ cells were seeded for expansion in 12-well culture plates (NUNC) at 2×10⁵ cells/ml. Cultures were established in RPMI1640 (GIBCO) medium containing 10% heat-inactivated FBS either in the presence of TNF-α (100 U/ml, Endogen) plus IL-3 (100 U/ml, Endogen) for CD14⁺ DCs or in the presence of TNF-α (100 U/ml) plus GM-CSF (500 U/ml, LG Chem, Taejon, Korea) for CD1a⁺ DCs. Optimal conditions were maintained by splitting these cultures every two days with medium containing fresh factors at a cell density of 2×10⁵ cells/ml. Cultures of 8 days and 18 days, respectively for immature and mature non-adherent DCs, were collected, stained with anti-CD1a-PE and anti-CD14-FITC (Becton Dickinson) and then sorted for single positive cells using either FACSCalibur or FACSort (Becton Dickinson).

CD11c⁻ DCs were isolated from peripheral blood using either a BDCA-4 Cell Isolation Kit (Miltenyi Biotec) or a Blood Dendritic Cell Isolation Kit with some modifications (Miltenyi Biotec) as follows: T cells, NK cells and monocytes were depleted from PBMC using haptenized anti-CD3, CD11b, and CD16 antibodies and anti-Hapten Microbeads. CD11c⁺ cells were then excluded from the flowthrough using anti-CD11c antibodies (Pharmingen) and goat anti-mouse microbeads (Miltenyi Biotec). CD4⁺/CD11c⁻ blood dendritic cells were then positively selected from the nonmagnetic fraction using MACS CD4 microbeads and Minimacs separation columns (Miltenyi Biotec). For matured DCs, freshly isolated CD11c⁻DCs were cultured for 5 days in RPMI 1640 medium supplemented with 10% autologous human serum (to avoid serum antigen-mediated unwanted stimulation), 200 U/ml of IL-3 (Endogen) and 5 μg/ml of human recombinant CD40L (raised in the inventors laboratory).

T lymphocytes were purified from PBMC by immunoaffinity depletion using T cell isolation kit (Pierce). B lymphocytes were obtained from the whole blood using RossettSep in accordance with the manufacturer's instruction (StemCell Technologies). Monocytes were purified from PBMC by adherence to the human gamma globulin-coated petri dishes.

Monocytes-derived DCs (MoDCs) were generated from adherent mononuclear cells. PBMCs were seeded in 6-well culture plates, at a density of 5×10⁶ cells/ml, allowed to adhere for 1 h at 37° C. and non-adherent cells were washed away with pre-warmed RPMI1640. Adherent cells were cultured for 7 days in RPMI 1640 medium supplemented with 10% autologous human serum and 1000 U/ml each of IL-4 (Endogen) and GM-CSF (LG Chem, Taejon, Korea). Media were refreshed at day 3 and 5. At day 7, non-adherent cells were collected as immature MoDCs by moderately vigorous agitation. For matured MoDC, non-adherent cells of day 7 were additionally cultured for 2 days in monocyte-conditioned medium (final concentration 50%, v/v) supplemented with 10 ng/ml of TNF-α (Pharmingen). The dead cells and contaminating lymphocytes were removed by Nycodenz density gradient centrifugation (34). In order to get CD1a⁻ MoDCs at day 9, autologous human serum was deliberately used as culture (22).

Total RNA was extracted from each subset of DC using Trizol reagent (Life Technologies, Inc) and mRNA was purified through affinity chromatography using polyATtrack system (Promega).

Generation of Subtractive DC-cDNA Library

cDNAs were synthesized as reported previously (35,36) using 200 unit of Superscript II (Life Technologies, Inc) and 200 ng of total RNA extracted from DC subsets and leukocytes. The first strand cDNA was prepared by mixing 200 ng of total RNA, 10 pmol of CDS primer (AAGCAGTGGTAACAACGCAGAGTACT30N_{—}_{¹}N,N=A, C, G, T; N_{—}_{¹}=A, C, G) and 10 pmol of SMART primer (AAGCAGTGGTAACAACGCAGAGTACGCGGG), cooling after heating at 70° C. for 2 min and adding Superscript H reverse transcriptase followed by incubation at 42° C. for 1 hr. The first strand cDNA thus prepared was diluted 5 times with TE buffer (10 mM Tris, pH 7.6, 1 mM EDTA) and the second strand cDNA was synthesized using a mixture of 6 μl of the diluted solution and PCR primer (AAGCAGTGGTAACAACGCAGAGT) by PCR (the total volume of 100 μl; 1 min at 95° C., 20-25 cycles [5 sec at 95° C., 5 sec at 65° C. and 6 min at 68° C.]).

Subtraction was performed in essence as described in the PCR-Select cDNA Subtraction kit (Clontech). In this subtraction, dendritic cells (CD1a⁺, CD14⁺, and CD11c⁻ DC) were used as tester and B cells, monocytes and T cells (BMT) were used as driver.

To overcome the limitation of DC supply, DC cDNA was pre-amplified using SMART PCR cDNA synthesis kit (Clontech). Similarly, BMT-cDNAs were pre-amplified in parallel. Amplified cDNAs were mixed either as tester (DC-cDNAs) amplicon or as driver (BMT-cDNAs) amplicon, and went through RsaI digestion and adaptor ligation, sequentially. The nucleotide sequences of the used adaptors are as follows:

| | |
|---|---|
| adaptor 1 | (5′-CTAATACGACTCACTATAGGGCTCGAGCGGCCGCCCGGGCAGGT-3′, |
| | 3′-GGCCCGTCCA-5′), |
| adaptor 2R | (5′-CTAATACGACTCACTATAGGGCAGCGTGGTCGCGGCCGAGGT-3′, |
| | 3′-GCCGGCTCCA-5′). |

Subtractive hybridization was performed twice in a 30-fold molar excess of driver over tester to remove cDNAs shared with BMT, resulting in enrichment of DC-specific cDNAs. First subtractive hybridization was performed with hybridization sample 1 and hybridization sample 2, respectively. Hybridization sample 1 was prepared through mixing adaptor 1-ligated tester DNA (10 ng) and 300 ng of driver DNA and adding 4× hybridization buffer. Hybridization sample 2 was prepared through mixing adaptor 2R-ligated tester DNA (10 ng) and 300 ng of driver DNA and adding 4× hybridization buffer. Both of the hybridization samples were denatured at 98° C. for 90 sec and hybridized at 68° C. for 8 hr. After adding 200 ng of driver DNA to both of the hybridization samples, the resultants were further hybridized at 68° C. overnight. Through such hybridization process, the genes not present in driver remain and thus can be amplified by PCR. Reverse-subtraction was performed in the same manner as described above except interchanging driver with tester.

Remaining cDNA after subtraction was selectively amplified by the first PCR for 27 cycles and then by the second nested PCR for 12 cycles. Subtracted cDNA was inserted into pGEM-T Easy vector (Promega) and transformed into E. coli DH5α to generate DC-specific subtracted cDNA library.

Colony PCR and Microarray Fabrication

Colonies were randomly selected from the subtracted DC-cDNA library and grown for 3 hr in LB for colony PCR. In addition, for microarray fabrication, 124 genes including CD (cluster of differentiation) and cytokine genes were purchased from Incyte (all Incyte's clones were sequenced) and additional 57 CD genes were PCR-amplified in the present inventor's laboratory and cloned into the T vector. Three plant genes, agpL (AF184598), agpS (AF184597), and mt45 (AF320905) were included as spike genes. The total of 2,304 clones was cultured in 96-well plates for PCR. PCR amplifications were performed in 100 μL volume with amidated vector-specific primers (lab1 5′-GTGCTGCAAGGCGATTAAG-3′, lab2 5′-GGAATTGTGAGCGGATAAC-3′) for 30 cycles of denaturation for 30 sec at 94° C., annealing for 30 sec at 62° C. and extension for 1.5 min at 72° C. Amplified DNAs were dissolved in 3×SSC, and then printed on microarrays with Q-bot (Genetix, UK). A DC microarray comprised of the subtracted DC cDNA library was fabricated for screening the DC-associated genes. Those DC-associated genes were mounted on another microarray in duplicate, named HI380, for revealing the DC subset-specificity and the effects of maturation and donor differences.

Microarray Analysis

The forward- and the reverse-subtracted amplicons (cDNA) were used for DC/BMT differential screening. Amplified cDNA of each DC subset was also used without subtraction as a probe for HI380 microarray analysis. A mixture of 1 μg of the cDNA and 20-100 pg of the plant spike DNAs was fluorescently labeled with either Cy3 or Cy5 dye by the random priming method using Klenow fragment (NEB) and random octamer. The labeled amplicon was purified through ethanol precipitation at a room temperature with two volumes of ethanol and resuspended in 40 μl of 4×SSC, 0.2% SDS, 0.1 μg/μl poly(dA), 0.1 μg/μl yeast tRNA, and 0.25 μg/μl Cot1 DNA. The labeled DNA was denatured at 100° C. for 5 min and then applied to the microarray for hybridization at 55° C. for 12-16 hr, followed by several washing steps. Fluorescent images of hybridized microarrays were obtained using a Scanarray 4000 microarray scanner (GSI Lumonics) and images were analyzed with GenePix Pro 3.0 (Axon Instruments). PMT and laser value for scanning were tuned by equalizing the intensities of Cy3 and Cy5 on a spike gene. Fluorescence ratios were calibrated by applying normalization factors calculated from the mean intensity of spike genes (over 6 spots on each microarray).

Back-Hybridization

Back-hybridization was performed to screen out redundant clones on microarray. Redundant clones revealed in sequencing analysis were PCR-amplified with primers flanking T-vector insertion site (sense 5′-TGCTCCCGGCCGCCAT-3′, antisense 5′-CGGCCGCGAATTCACTAG-3′). Redundant clones (Ig superfamily protein, MHC class II DR pool, mitochondrial gene pool, osteopontin, annexin A2, MMP-12, and α-tublin) were collected, labeled with Cy3 or Cy5, and hybridized with the microarray. To minimize the background hybridization between vector sequences, single strand DNA was included in hybridization reaction as a blocking DNA. The single strand DNA was prepared by asymmetric PCR with lab1 primer using self-ligated pGEM T-Easy PCR product (lab1 and lab2 primed) as a template. Redundant clones showing the intensity value higher than 10,000 were screened out.

Sequence Analysis

Selected clones on microarray were recovered from cell stock and each insert in pGEM T-Easy was amplified with M13 forward and reverse primers located inside of lab1 and lab2 primers. The PCR products were sequenced with the Big Dye terminator kit (Perkin-Elmer) and analyzed with a 377 ABI automated 96-lane sequencer (Perkin-Elmer). Around 200-700 bp sequences were trimmed for vector sequence with Seqman 4.03 (DNASTAR Inc.) and were analyzed with Advanced BLAST search (http://www.ncbi.nlm.nih.gov/BLAST/). The chromosomal location was also examined by BLAT search (http://genome.ucsc.edu).

Quantitative PCR

The initial cDNA content in each sample was normalized with the amount of GAPDH. Quantitative PCR amplifications were performed in a 50 μl volume using 4 ng of each cDNA on Perkin-Elmer DNA thermocycler 9600 Prism for 30 cycles (15 sec at 94° C., 20 sec at 55° C. and 1 min at 72° C.). To evaluate the specificity of each message semi-quantitatively, 10 μL each of the PCR products were withdrawn from 25 cycles and from 30 cycles, respectively, and then run simultaneously on 1.1% agarose gels. PCR primers to each selected clone were designed with PrimerSelect 4.03 (DNASTAR Inc.). The expected sizes of PCR products were 300 to 600 bp and the optimal annealing temperature was 55 to 65° C.

Results

Immunophenotypes of Purified Dendritic Cells (DC)

The purity of CD1a⁺ DCs CD14⁺ DCs, or CD11c⁻ DCs for the construction of a subtractive DC-cDNA library was 90±4%, and the purities of each DC subsets in the additional experiments were over 98% after cell sorting or isolation (FIG. 1a). CD1a⁺ DCs and CD14⁺ DCs at day 18 were strikingly distinguished, not only by their surface phenotypes, but also by their morphologies (FIG. 1b). However, these two DCs were very similar in their levels of HLA-DR, CD83 and CD86 expression. The expression of DC-Lamp was observed only in CD1a⁺ DCs but not in CD14⁺ DCs at day 18. In this specific batch of CD14⁺ DCs, the up-regulation of CD83 was observed in parallel, with an unusual decrease in their levels of HLA-DR and CD86, during their development between day 8 and 18. While Langerin staining was expected in CD1a⁺ DCs between day 8 and 12 (37), CD1a⁺ DCs at day 8 were not stained by Langerin mAb (monochlonal antibody), probably indicating the kinetic variation between different cultures of CD1a⁺ DCs. The expression of Lagerin at day 18 in CD1a⁺ DCs was not apparent in their immunostaining, although the microarray analysis on the same DCs revealed the up-regulated Langerin expression at day 18 in CD1a⁺ DCs (Table 2). Thus, CD1a⁺ DCs at day 18 were considerably not in the window of the Lagerin⁺ phenotype during their maturation (37). Unlike these cytokine-induced DCs, CD11c⁻ DCs freshly isolated from peripheral blood barely expressed CD86 and were relatively small and even in size (FIG. 1b). On the other hand, CD11c⁻ DCs at day 5, expressed significant levels of CD86, as well as of CD80 and DC-Lamp. The representative phenotypes of MoDCs were CD1a⁺/CD83⁻/DC-Lamp⁻ at day 7 and CD1a⁻/CD83⁺/DC-Lamp⁺ at day 9. Depending on donor, there was some degree of differences, but always within the allowable range, in the level of expression, among the DCs of the same subset.

High Specificity of Subtractive DC-cDNA Library to DC Subsets

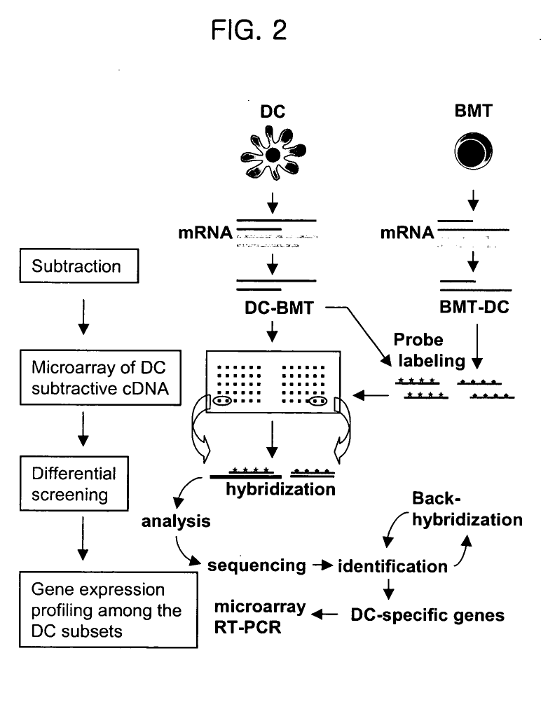

In order to gain direct access DC specific genes without being hampered by highly abundant messages shared by most leukocytes, the present inventors have employed DC cDNA subtraction strategy followed by microarray analysis (FIG. 2). DC subtractive library was constructed by subtracting B cells, monocytes, and T cells messages concurrently from the combined ones of CD1a⁺, CD11c⁻, and CD14⁺ DCs. In this subtraction, the present inventors carried out the modified subtractive hybridization, termed PCR-select (38), which exploits suppressive PCR to selectively enrich the subtracted genes. To compare the specificity of subtraction, not only forward (DC subtracted by BMT) but also reverse subtraction (BMT subtracted by DC) was performed in parallel. The profile of PCR products from either subtraction revealed a unique pattern of discrete bands on agarose gel that were absent in non-subtracted control. To examine the integrity of DC subtractive library, eight clones were randomly selected and sequenced. Sequence analysis revealed two cDNAs that correspond with the sequence from MMP12 gene, three cDNAs of mitochondrial genes, and two novel ESTs. The high efficacy of subtraction was indicated by the absence of cDNAs corresponding to well-known housekeeping genes among theses randomly picked clones. To further assess the integrity of subtraction, the forward and reverse subtracted cDNAs were labeled with Cy3 and Cy5, respectively, and then hybridized on a microarray with 2000 known genes (generous gift from Dr. Park JH at KRIBB, Taejon, Korea) in double blinded approaches. Most of the spots developed single fluorescence of either Cy3 or Cy5 and few developed fluorescence of both Cy3 and Cy5. These results suggest that the subtraction was successfully performed for the depletion of the common messages in two cDNA populations, so that the subtracted cDNA was acceptable as a specific probe for each population in the following microarray analysis.

Novel DC-Associated Genes Identified by Microarray Analysis

To identify DC-associated genes, 1,920 clones from a DC subtracted library, and 181 cDNAs of CD and cytokine genes were immobilized on a glass slide and subjected to differential hybridization using cDNA probes manipulated as follows: The forward-subtracted (DC-specific) and the reverse-subtracted (BMT-specific) cDNAs were labeled differentially with Cy3 or Cy5 and then co-hybridized with the cDNAs on the same microarray. To normalize the intrinsic signal differences coming from Cy3 and Cy5 labeling, another hybridization was set up for reverse labeling with Cy3 and Cy5. As was expected, quick visual inspection of hybridization signals revealed that the majority of the spots originating from the DC subtracted library were DC specific, so that they were not detected among the dual-labeled ones but strongly hybridized with DC-specific probes. In contrast, most of the known CD genes were not DC-specific in the sense that they were barely detected with DC-specific probes, and only a few were strongly labeled by BMT-specific probes.

Of the 1,920 clones, 1,140 were selected for their propensity to adopt highly DC-specific signals (threshold intensity ratio of DC/BMT>3). To minimize the number of clones to be analyzed, redundant clones had to be screened out. For this purpose, 74 clones were randomly selected and sequenced. Of the 74 clones sequenced, 31 were unique genes. The following genes were most frequently identified: Ig-superfamily, mitochondrial genes (COI and COIII, 12S rRNA, 16S rRNA and cytochrome b), MHC class II DR alpha, Matrix metalloprotease 12 (MMP-12), Osteopontin (Eta-1), Annexin A2 and α-Tublin. Since the combined redundancy of these clones comprised 62% of the sequenced clones, back-hybridization was performed using a pool of these genes to screen out redundant clones. Thereby, the 300 cDNA clones were sequenced and searched by BLAST for gene identification. Finally, these analyses revealed 69 non-overlapping genes. Of these, 63 genes were found to encode known proteins and six were novel sequences, which had no matches in GenBank database but some of them had matches in human genome sequence (Table 1). The specificity of each clone DC was designated as the ratio of DC/BMT signal intensity obtained from differential hybridization. It appeared that some of them were still more frequently identified than others even after screening-out the redundant ones. It was noted that Ig superfamily protein (Z23IG) was not only highly DC-specific but also apparently abundant among the DC-associated messages. In addition to the genes reported previously in association with MoDCs (26, 28 and 29), the present invention revealed new members of DC-associated genes including Ig superfamily protein (Z39IG), CD20-like precursor, Glycoprotein nmb (GPNMB), TGFβ-induced protein (TGFBI), Myeloid DAP12-associated lectin (MDL-1), and the six no-match genes.

The six no-match genes were named “Crea 2, Crea 7, Crea, 11, Crea 12, Crea 13, Crea 14”, respectively and their nucleotide sequences are shown in SEQ ID NOs:1-6, respectively.

Expression Profile for DC-Associated Genes in DC Subset

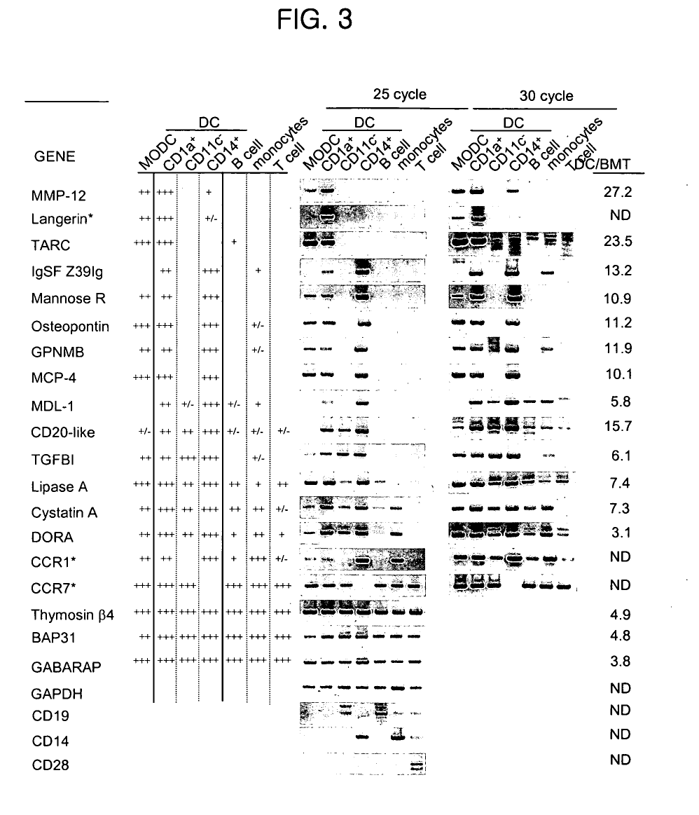

The DC-associated genes identified in the DC/BMT differential microarray analysis were then further examined for their expression profiles in four different DC subsets, and other leukocytes, using another microarray, HI380 (Table 2) and semi-quantitative RT-PCR (FIG. 3). Some other genes, such as CCR1, CCR7, DC-Lamp, E-cadherin, Langerin, etc, were also included in the subset-specific expression analysis. As shown in Table 2 and FIG. 3, the result from microarray were in good agreement with the results revealed in the quantitative RT-PCR.

As expected from lineage differences, the most striking difference was seen between CD11c⁻ lymphoid DCs and the ex vivo generated myeloid DCs. Most of the DC-associated genes identified from the primary microarray analysis, such as α- and β-Tubulin, Osteopontin (Eta-1), Glycoprotein nmb (GPNMB), MCP4, Lysosomal acid lipase, Enolase 1, Thymosin β4, Ferritin L-chain, Annexin A2, VAMP8 and GABARAP, were not highly expressed in CD11c⁻ DCs (Table 2 and FIG. 3). On the other hand, Interferon regulatory factor 4 (IRF-4), which is essential for mature T and B cell function (39), was highly expressed in CD11c− DCs. CD11c− DCs are the main producers of Type I Interferon in human blood (40-43). Besides IRF4, dihydropyrimidinase related protein-2 (DRP-2) and 5-lipoxygenase activating protein (FLAP) were markedly detected in CD11c⁻ DCs.

While the difference was not as remarkable as that shown in CD11c⁻ DCs, there were some differences in the expression profiles of DC-associated genes among the three myeloid DCs (CD1a⁺ DCs, CD14⁺ DCs and MoDCs) (Table 2 and FIG. 3). For example, TARC, IRF4, CCR7, and DC-Lamp were highly expressed in two DCs, MoDCs and CD1a⁺ DCs. However, in CD14⁺ DCs, the expression levels of these genes were comparable to those of the non-DC populations. Meanwhile, MCP1, Eta-1, TGFBI, Factor XIIIa, Mannose receptor, Ig superfamily (Z39IG) etc. were relatively prominent in CD14⁺ DCs. Lastly, while the expressions of S100B, MMP-12 and CD1b, were remarkable in CD1a⁺ DCs, these genes were not seemingly specific for CD1a⁺ DCs. The relatively lower expression of these genes in MoDCs at day 9 were likely to reflect the differences of the DCs in the acquired degree of maturation, based on the previous findings of their association with the maturity of MoDCs (26-29,39). It's not clear why, but certain genes, such as Thymosin β4, BAP31 and GABARAP, showing a relatively lower DC/BMT ratio (<6.0), failed to reveal the DC-specificity in the quantitative RT-PCR analysis (FIG. 3). Nevertheless, considering the report that DORA, a gene showing the lowest ratio (3.1) of DC/BMT in this experiment (Table 1 and FIG. 3), was screened to be DC-specific in another independent study, the genes in this range of the DC/BMT ratio in this experiment also seemed necessary to be counted in for further consideration. Human myeloid DCs are developed from various sources, including blood monocytes and CD34⁺ hematopoietic stem cells. Likely expected from different precursors under different tissue-cytokine microenvironments, the three DC subclasses of myeloid origin (CD1a⁺ DCs, CD14⁺ DCs and MoDCs), appeared to be somewhat different in their gene regulations. According to the data shown in Table 2, the differences in these myeloid DC subclasses were in the range of “from the subtle to the profound”, indicating the presence of shared as well as unique functions. The most outstanding difference was revealed in their expression levels of chemokine and chemokine receptors, implying their remarkable differences in the trafficking property. Based on the previous findings (49), MoDCs and CD1a⁺ DCs showing the unique up-regulations of TARC and CCR7 could be identified as authentic migratory DCs, traveling from tissue to draining lymph nodes to convey Ag to T cells. CD14⁺ DCs had features of tissue resident DC with a transcription profile of “no CCR7 but more CCR1” and “no TARC but more MCP-1 and Eta-1”.

Summarizing those described above, genes highly expressed in myeloid DCs are those involved in antigen-uptake/processing/presentation, cell metamorphosis, or chemotaxis. Most of the genes previously identified in MoDCs, such as TARC, Ferritin L-chain, Lysosomal acid lipase, α- and β-Tubulin, Osteopontin (Eta-1), etc., are not markedly expressed in CD11c⁻ DCs, regardless of their maturation status. On the other hand, specific transcription factors and MHC class II molecules, e.g., Interferon regulatory factor 4 (IRF4), HLA-DR, are similarly expressed in both myeloid DCs and CD11c⁻ DCs.

Expression of DC-Associated Genes in Different Maturation Stages

To answer the question how the differentiation/maturation stage of DC affects the difference in DC-associated gene expression, the inventors undertook similar microarray analysis for the immature myeloid DCs and the matured CD11c⁻ DCs (Table 3). While matured by culturing for 5 days under the influence of CD40L and IL-3, CD11c⁻ DCs did not over-express the messages commonly up-regulated in fully differentiated DCs of myeloid origin. Thus, these genes such as α- and β-Tubulin, Eta-1, GPNMB, MCP4, Lysosomal acid lipase, Enolase 1, Thymosin β4, Ferritin L-chain, Annexin A2, VAMP8 and GABARAP, were considered to be truly myeloid DC-associated ones. Interestingly, the high expression of FLAP, implicated in allergic inflammation (44), were not restricted to certain stage of maturation and relatively common to the DC subclasses including CD11c⁻ DCs. The exceptional absence of the FLAP over-expression in MoDCs at day 9 was repeatedly observed. The IRF4 expression in CD1a⁺ DCs, CD11c⁻ DCs and CD14⁺ DCs was markedly down-regulated upon the DC maturation, but was completely reversed in MoDCs (Table 3), suggesting that the control of IRF4 expression seems to be cell type-specific, even among the myeloid DCs. However, in the other set of experiments, the IRF4 expression was considerably high in CD1a⁺ DCs at day 18, suggesting that the control of the IRF4 expression may be relied upon the signals including the one related with maturation. The most of the DC-associated messages were not remarkable in immature DCs, and seemed to be associated with the maturity of the relevant DC subclasses. For example, MMP12, Z39IG, GPNMB, Eta-1 etc showed the DC/BMT ratio lower than 1 in the immature DCs. However, there were genes constitutively over-expressed in the immature stage of the relevant DCs (DC/BMT>1). These genes included TARC in MoDCs, MHC class II DRα in all of four DC subclasses, CD1b in MoDCs and CD1a⁺ DCs, CD20-like precursors and MRC1 in the two CD34⁺-derived DCs, Lysosomal acid lipase and TGFBI in MoDCs. For certain cases, the DC subset-specific expression profile was obvious from their early stage of DC development. Thus, the profound expression of MCP1 in CD14⁺ DCs, and the counterpart of DC-Lamp in CD1a⁺ DCs, were such cases, suggesting their own DC developments through truly distinct pathways from the same precursor.

The absence or the lower level of DC-Lamp expression has been described for the immature forms in MoDCs (54), CD1a⁺ DCs (55) and CD11c⁻ DCs (40). The result from microarray analysis of this invention indicated that DC-Lamp expression was absent in CD14⁺ DCs at any stage of their development. In good contrast to this, CD1a⁺ DCs appeared to up-regulate the DC-Lamp at as early as day 8 of the culture (FIG. 1b, Table 3). However, comparing the immuno-phenotypes of CD14⁺ DCs (day 18) with those of CD1a⁺ DCs (day 8), it seems not appropriate to consider CD14⁺ DCs (day 18) as less mature than CD1a⁺ DCs (day 8). In this regard, the data of this experiment support the consideration of CD14⁺ DCs as distinct DCs derived from the common myeloid precursor.

TGFβ dependency of LC development is well documented both in vivo (56) and in vitro (57,58). CD1a⁺ DCs were possible to be developed not only from the myeloid precursor but also from other types of myeloid DCs, including blood CD11c⁺ DCs, CD14⁺ DCs and MoDCs with the provision of TGFβ in culture system (58,59,60)□ Finding of TGFβ induced protein (TGFBI) among the DC-associated genes seems to insinuate the endogenous production of TGFβ, supporting CD1a⁺ DC development to a certain degree in almost any culture of myeloid DCs. In addition to TGFBI, the present invention identified new members of DC-associated genes. These included Ig superfamily protein (Z39IG) (62), Glycoprotein nmb (GPNMB) (63), CD20-like precursor (64), and Myeloid DAP12-associated lectin (MDL-1) (65). While these genes are seemingly important in DC biology, for encoding membrane proteins at the cell surface and a secretary protein, it is almost uncertain where to put them in the context of DC functions.

The up-regulation of some DC-associated genes is likely to explain the connection of DC subtypes with certain human diseases. These genes include high affinity IgE receptor a (FcERI) and CD36 of CD1a⁺ DCs in atopic dermatitis (66), FLAP of CD11c⁻DCs in human nasal allergy (67), and Eta-1 of CD14⁺ DCs in erythema elevatum diutinum.

Expression of DC-Associated Genes in Different Donors

To examine how donor difference affect the results, the present inventor prepared another set of four DC subclasses from different donors and undertook microarray analysis with the cDNA probes freshly derived from the second set of DCs. Most of the representative genes showed a “relative consistency” in general for their expression profiles in different donors. The “relative consistency’ was found for the expression of TARC, Ig superfamily (Z39IG), MCP1, TGFBI, CCR1, DC-Lamp, E-cadherin and DEC205. However, the “relative consistency” for the expression of Eta-1, MRC1 and IRF4, was not so strong as those mentioned above. Among the DC-associated genes newly identified, MDL-1 was also consistent in a different donor set.

TABLE 1

| Gene | | GenBank | Redundancy | Intensity of | Intensity of | |
|---|---|---|---|---|---|---|
| No, | Name | accession no. | In screening | BMT | DC | DC/BMT |
| 1 | Alpha-tubulin* | K00558 | 4 | 1312 | 37669 | 28.7 |
| 2 | S100 calcium-binding protein, beta (S100B) | NM_006272 | 2 | 1036 | 28546 | 27.6 |
| 3 | Matrix metalloproteinase 12 (MMP12)* | XM_006272 | 5 | 1402 | 38171 | 27.2 |
| 4 | Thymus and activation-regulated chemokine (TARC) | D43767 | 1 | 1109 | 26047 | 23.5 |
| 5 | Myosin phosphatase, target subunit 1 (MYPT1) | XM_006578 | 1 | 2376 | 43787 | 18.4 |
| 6 | CD1B antigen (CD1B) | XM_002174 | 4 | 1286 | 20646 | 16.1 |
| 7 | CD20-like precursor | NM_022349 | 15 | 1746 | 27393 | 15.7 |
| 8 | Ig superfamily protein (Z39IG)* | XM_010265 | 23 | 1430 | 18815 | 13.2 |
| 9 | MHC class II HLA-DQ-alpha chain | U77589 | 1 | 1071 | 13284 | 12.4 |
| 10 | glycoprotein nmb (GPNMB) | XM_004781 | 3 | 1170 | 13914 | 11.9 |
| 11 | osteopontin* | NM_000582 | 6 | 2964 | 33178 | 11.2 |
| 12 | 5-lipoxygenase activating protein (FLAP) | M63262 | 6 | 1126 | 12316 | 10.9 |
| 13 | mannose receptor, C type 1 (MRC1) | NM_002438 | 3 | 1284 | 13960 | 10.9 |
| 14 | cytochrome b-245, beta polypeptide (CYBB) | NM_000397 | 3 | 1050 | 11226 | 10.7 |
| 15 | monocyte chemotactic proteins 4 (MCP4; SCYA13) | XM_008411 | 4 | 1429 | 14456 | 10.1 |
| 16 | MHC class II HLA-DR-alpha chain* | XM_004209 | 8 | 1454 | 14578 | 10.0 |
| 17 | MHC class II HLA-DR-beta chain | M26038 | 6 | 1607 | 14838 | 9.2 |
| 18 | Rnase A family 1 (RNASE1) | XM_012375 | 2 | 2413 | 21588 | 8.9 |
| 19 | dihydropyrimidinase related protein-2 | D78013 | 4 | 1997 | 17249 | 8.6 |
| 20 | ATP synthase 6 (MTATP6; mitochondria) | AF347015 | 11 | 3394 | 26657 | 7.9 |
| 21 | 16S ribosomal RNA (mitochondria)* | AF347015 | 6 | 2683 | 21015 | 7.8 |
| 22 | lysosomal acid lipase | U08464 | 22 | 1043 | 7756 | 7.4 |
| 23 | Cystatin A (CSTA) | NM_005213 | 4 | 1286 | 9437 | 7.3 |
| 24 | Antigen CD36 | M98399 | 8 | 1404 | 9501 | 6.8 |
| 25 | Immunoglobulin transcription factor 2 (IFT2) | XM_012756 | 1 | 1719 | 11613 | 6.8 |
| 26 | monocyte chemotactic proteins 1 (MCP1; MCAF) | M24545 | 2 | 2311 | 14802 | 6.4 |
| 27 | beta-actin | BC004251 | 1 | 715 | 4561 | 6.4 |
| 28 | transforming growth factor, beta-induced, 68 kD (TGFBI) | NM_000358 | 2 | 1318 | 8002 | 6.1 |
| 29 | cytochrome b (MTCYB; mitochondria)* | AF347015 | 2 | 3660 | 21907 | 6.0 |
| 30 | calmodulin | D45887 | 3 | 752 | 4383 | 5.8 |
| 31 | Myeloid DAP12-associating lectin (MDL-1) | AJ271684 | 1 | 1345 | 7736 | 5.8 |
| 32 | cytochrome C oxidase II (COII; mitochondria) | AF347015 | 19 | 2555 | 14572 | 5.7 |
| 33 | beta-tubulin | J00314 | 1 | 1235 | 6826 | 5.5 |
| 34 | B cell linker protein (BLNK) | NM_013314 | 2 | 1615 | 8909 | 5.5 |
| 35 | Activated RNA polymerase II transcription cofactor 4 (PC4) | XM_011218 | 2 | 1980 | 10656 | 5.4 |
| 36 | cytochrome C oxidase I (COI; mitochondria)* | AF347015 | 4 | 2499 | 13410 | 5.4 |
| 37 | enolase 1, alpha (ENO1) | NM_001428 | 1 | 520 | 2607 | 5.0 |
| 38 | 90 kDa heat shock protein (hsp90) | M16660 | 1 | 2040 | 10213 | 5.0 |
| 39 | interleukin 2 receptor, gamma | NM_000206 | 1 | 1161 | 5741 | 4.9 |
| 40 | thymosin, beta 4, X chromosome (TMSB4X) | NM_021109 | 5 | 1649 | 8138 | 4.9 |
| 41 | Ferritin, light polypeptide | BC004245 | 3 | 2527 | 12327 | 4.9 |
| 42 | NADH dehydrogenase subunit 4 (MTND4; mitochondria) | AF347015 | 1 | 3738 | 18023 | 4.8 |
| 43 | accessory proteins BAP31/BAP29 | NM_005745 | 5 | 966 | 4598 | 4.8 |
| 44 | Annexin A2 (ANXA2)* | NM_004039 | 8 | 1221 | 5506 | 4.5 |
| 45 | isocitrate dehydrogenase 3 (NAD⁺) alpha (IDH3A) | XM_007580 | 1 | 1754 | 7766 | 4.4 |
| 46 | microsomal glutathione S-transferase 2 (MGST2) | XM_003461 | 1 | 1540 | 6767 | 4.4 |
| 47 | 12S ribosomal RNA (mitochondria)* | AF347015 | 4 | 1439 | 6317 | 4.4 |
| 48 | cytochrome C oxidase III (COIII; mitochondria)* | AF347015 | 11 | 1913 | 8318 | 4.3 |
| 49 | coagulation factor XIII, A1 polypeptide | AK001685 | 1 | 1009 | 4225 | 4.2 |
| 50 | GABA(A) receptor-associated protein (GABARAP) | NM_007278 | 2 | 1221 | 4666 | 3.8 |
| 51 | serine/threonine kinase 15 | BC002499 | 1 | 1369 | 5048 | 3.7 |
| 52 | Vesicle-associated membrane protein 8 (VAMP8) | XM_002561 | 2 | 1976 | 7267 | 3.7 |
| 53 | Nicastrin | AF240468 | 1 | 1359 | 4997 | 3.7 |
| 54 | purinergic receptor (family A group 5) | XM_007212 | 1 | 1592 | 5833 | 3.7 |
| 55 | Rho GDP dissociation inhibitor beta (ARHGDIB) | NM_001175 | 1 | 1596 | 5707 | 3.6 |
| 56 | MAD homolog 2 (MADH2) | XM_008795 | 1 | 719 | 2523 | 3.5 |
| 57 | cytochrome c, clone MGC: 12367 | BC005299 | 1 | 1330 | 4538 | 3.4 |
| 58 | MHC class II HLA-DM-alpha chain | XM_004220 | 1 | 1403 | 4552 | 3.2 |
| 59 | high affinity IgE receptor alpha-subunit (FcERI) | X06948 | 1 | 116 | 369 | 3.2 |
| 60 | MLN51 | X80199 | 1 | 1405 | 4431 | 3.2 |
| 61 | DORA protein | AJ223183 | 1 | 1411 | 4341 | 3.1 |
| 62 | GTP-binding protein (NGB) | XM_005722 | 1 | 877 | 2674 | 3.0 |
| 63 | interferon regulatory factor 4 (IRF4) | NM_002460 | 1 | 565 | 1680 | 3.0 |
| 64 | Crea2 | NA | 2 | 1437 | 12646 | 8.8 |
| 65 | Crea7 | NA | 5 | 2947 | 15620 | 5.3 |
| 66 | Crea11 | NA | 1 | 2639 | 8446 | 3.2 |
| 67 | Crea12 | NA | 1 | 1449 | 4928 | 3.4 |
| 68 | Crea13 | NA | 1 | 1336 | 9218 | 6.9 |
| 69 | Crea14 | NA | 1 | 2201 | 14304 | 6.5 |

Table 1 is a list of DC-associated genes identified from subtraction, microarray and sequence analysis. DC/BMT represents the ratio of fluorescence intensity as determined by forward (DC-BMT) and backward (BMT-DC) subtracted probes. The symbol (*) indicates the DNA clones used for the screening-out experiments to minimize the number of redundant clones. NA; not applicable.

TABLE 2

| | | Ratio of DCs and BMT (DCs/BMT) | | | | | |
|---|---|---|---|---|---|---|---|
| Gene | | | | MoDC | CD1a⁺DC | CD11c⁻DC | CD14⁺DC |
| No. | Name | BMT intensity | CD34⁺ cells | Day 9 | Day 18 | Day 0 | Day 18 |
| 1 | Alpha-tubulin | 13617 | 0.2 | 2.9 | 2.9 | 0.2 | 1.7 |
| 2 | S100B | 3416 | 0.7 | 0.9 | <28.6> | 0.7 | <5.6> |
| 3 | MMP12 | 1420 | 0.9 | 0.7 | <4.9> | 0.5 | 0.3 |
| 4 | TARC | 4113 | 0.5 | <20.0> | <24.0> | 0.6 | 0.8 |
| 5 | MYPT1 | 3805 | 0.4 | 1.0 | 2.0 | 0.4 | <4.9> |
| 6 | CD1B | 3107 | 0.4 | 2.4 | <17.6> | 0.8 | 1.5 |
| 7 | CD20-like | 3171 | 0.4 | 0.8 | <18.0> | 1.7 | <20.7> |
| 8 | Ig_superfamily Z391G | 2016 | 0.4 | 0.9 | 2.9 | 0.6 | <16.4> |
| 9 | MHC DQ-alpha | 11817 | 0.2 | 3.2 | <9.6> | 0.5 | 2.0 |
| 10 | GPNMB | 3290 | 0.5 | 1.7 | 1.2 | 0.8 | <5.0> |
| 11 | Eta-1 | 2409 | 0.3 | 3.2 | <6.3> | 0.5 | <11.8> |
| 12 | FLAP | 8060 | 0.3 | 0.9 | <8.1> | 3.0 | <5.4> |
| 13 | MRC1 | 2097 | 0.5 | 1.6 | 3.8 | 0.7 | <6.1> |
| 15 | MCP4 | 3454 | 0.3 | 2.9 | <4.5> | 0.4 | <5.6> |
| 16 | MHC DR-alpha | 22066 | 0.7 | 2.2 | 3.8 | 2.4 | 1.3 |
| 17 | MHC DR-beta | 20288 | 0.2 | 0.7 | 2.7 | 1.2 | 1.4 |
| 18 | RNasel | 3684 | 0.2 | 1.6 | 1.1 | 0.4 | <11.5> |
| 19 | DRP-2 | 1981 | <6.1> | <8.9> | 2.4 | <5.9> | 1.1 |
| 22 | lysosomal acid lipase | 2801 | 0.3 | <4.2> | <8.1> | 0.3 | <6.1> |
| 23 | cystatin A | 3317 | 0.2 | 0.8 | <17.4> | 0.3 | <4.3> |
| 24 | CD36 | 1651 | 0.4 | 0.8 | 2.9 | 0.8 | 1.2 |
| 25 | ITF2 | 4145 | 0.4 | 1.1 | 0.8 | 1.2 | 1.3 |
| 26 | MCP1 | 10899 | 0.1 | 0.7 | 0.5 | 0.2 | <9.6> |
| 28 | TGFBI | 3179 | 0.4 | 1.8 | 3.4 | 1.5 | <9.2> |
| 30 | Calmodulin | 6950 | 0.9 | <5.1> | 1.8 | 0.4 | 1.6 |
| 31 | MDL-1 | 3245 | 0.3 | 1.0 | 1.3 | 0.6 | 1.9 |
| 33 | beta-tubulin | 12754 | 0.3 | 2.8 | 2.8 | 0.3 | 2.7 |
| 34 | BLNK | 6112 | 0.2 | 0.5 | 0.8 | 0.4 | 0.5 |
| 35 | PC4 | 9653 | 0.2 | 0.4 | 0.9 | 0.2 | 0.4 |
| 37 | enolase 1 | 14759 | 0.1 | 3.8 | 1.4 | 0.1 | 1.5 |
| 38 | hsp90 | 8933 | 0.2 | 0.6 | 1.1 | 0.3 | 0.8 |
| 39 | IL2Rg | 5573 | 0.3 | 0.5 | 0.7 | 0.2 | 0.2 |
| 40 | thymosin, beta 4 | 19186 | 0.3 | 1.9 | 3.4 | 0.1 | 1.6 |
| 41 | Ferritin | 31033 | 0.1 | 3.7 | 1.9 | 0.0 | <5.1> |
| 43 | BAP31/BAP29 | 9919 | 0.6 | 1.0 | 1.1 | 0.4 | 1.3 |
| 44 | annexin A2 | 12178 | 0.1 | 3.1 | <4.8> | 0.1 | 2.2 |
| 45 | IDH3A | 2298 | 0.4 | 0.6 | 0.6 | 0.9 | 0.5 |
| 46 | MGST2 | 3060 | 0.3 | 1.0 | 3.5 | 0.5 | 3.5 |
| 49 | Factor XIII | 2110 | 0.3 | 1.0 | 0.7 | 0.5 | 3.0 |
| 50 | GABARAP | 9150 | 0.1 | 1.4 | 1.3 | 0.2 | 2.0 |
| 51 | Serine/threonine kinase 15 | 2595 | 0.4 | 1.3 | 1.6 | 0.7 | 1.4 |
| 52 | VAMP8 | 9344 | 0.1 | 2.8 | <5.1> | 0.3 | <4.2> |
| 53 | Nicastrin | 3652 | 0.3 | 0.9 | 0.7 | 0.6 | 0.7 |
| 54 | purinergic receptor | 1901 | 0.4 | 1.0 | 3.1 | 0.6 | 1.9 |
| 55 | ARHGDIB | 23059 | 0.1 | 0.2 | 1.0 | 0.1 | 2.0 |
| 56 | MAD homolog 2 | 2167 | 0.5 | 0.9 | 0.8 | 0.6 | 0.7 |
| 58 | MHC DM-alpha | 8207 | 0.4 | 1.9 | <4.1> | 0.6 | 2.8 |
| 59 | FcERI | 2989 | 0.3 | 0.7 | 1.9 | 0.3 | 0.7 |
| 60 | MLN51 | 15001 | 2.2 | 1.4 | 0.9 | 1.9 | 1.4 |
| 61 | DORA | 5405 | 0.3 | 0.9 | <4.0> | 0.4 | 1.8 |
| 62 | GTP-binding protein | 3838 | 0.2 | 1.0 | 1.1 | 0.6 | 0.7 |
| 63 | IRF4 | 2005 | 0.5 | <7.9> | 2.2 | <9.3> | 0.4 |
| S1 | CCR1 | 2883 | 0.2 | 0.8 | 0.8 | 0.3 | 1.6 |
| S2 | CCR7 | 9952 | 0.1 | 3.1 | 1.5 | 0.1 | 0.1 |
| S3 | DC-Lamp | 3440 | 0.3 | <7.4> | <6.3> | 0.4 | 0.4 |
| S4 | E-cadherin | 3589 | 0.4 | 1.0 | 1.0 | 0.9 | 0.9 |
| S5 | DEC205 (CD205) | 2831 | 0.2 | 2.9 | 1.6 | 0.8 | 0.7 |
| S6 | Mannose receptor (CD206) | 2319 | 0.5 | 1.8 | <6.6> | 0.4 | <8.6> |
| S7 | Langerin (CD207) | 3482 | 0.3 | 1.2 | <5.9> | 0.8 | 1.0 |
| S8 | DC-sign (CD209) | 2305 | 0.3 | 1.0 | 0.8 | 0.4 | 0.9 |

Table 2 represents analysis of DC-associated gene expressions among the DC subsets with microarray. BMT intensity means the average signal intensity of B, Mc and T cells in 5 different experimental sets.

TABLE 3

| | | Ratio of DC and BMT | | | | | | | |
|---|---|---|---|---|---|---|---|---|---|
| Gene | | MODC | | CD1a⁺DC | | CD11c⁻DC | | CD14⁺DC | |
| No. | Name | Day 7 | Day 9 | Day 8 | Day 18 | Day 0 | Day 5 | Day 8 | Day 18 |
| 2 | S100B | 1.4 | 0.9 | 1.9 | 28.6 | 0.7 | 0.9 | 0.9 | 5.6 |
| 3 | MMP12 | 0.6 | 0.7 | 0.4 | 4.9 | 0.5 | 0.7 | 0.5 | 0.3 |
| 4 | TARC | 2.0 | 20.0 | 0.7 | 24.0 | 0.6 | 0.9 | 0.7 | 0.8 |
| 5 | MYPT1 | 0.6 | 1.0 | 0.6 | 2.0 | 0.4 | 0.4 | 1.0 | 4.9 |
| 6 | CD1B | 1.5 | 2.4 | 1.4 | 17.6 | 0.8 | 0.9 | 0.7 | 1.5 |
| 7 | CD20-like | 1.9 | 0.8 | 6.3 | 18.0 | 1.7 | 0.6 | 6.2 | 20.7 |
| 8 | Ig_superfamily Z39IG | 0.4 | 0.9 | 0.3 | 2.9 | 0.6 | 0.8 | 0.5 | 16.4 |
| 9 | MHC DQ-alpha | 1.2 | 3.2 | 0.8 | 9.6 | 0.5 | 0.5 | 1.0 | 2.0 |
| 10 | GPNMB | 0.4 | 1.7 | 0.7 | 1.2 | 0.8 | 0.7 | 1.0 | 5.0 |
| 11 | Eta-1 | 0.8 | 3.2 | 0.4 | 6.3 | 0.5 | 0.5 | 0.7 | 11.8 |
| 12 | FLAP | 2.7 | 0.9 | 6.8 | 8.1 | 3.0 | 4.9 | 7.0 | 5.4 |
| 13 | MRC1 | 0.9 | 1.6 | 2.1 | 3.8 | 0.7 | 1.0 | 1.2 | 6.1 |
| 15 | MCP4 | 1.1 | 2.9 | 0.3 | 4.5 | 0.4 | 0.6 | 1.2 | 5.6 |
| 16 | MHC DR-alpha | 2.6 | 2.2 | 3.5 | 3.8 | 2.4 | 3.3 | 3.9 | 1.3 |
| 17 | MHC DR-beta | 1.4 | 0.7 | 1.8 | 2.7 | 1.2 | 0.7 | 1.2 | 1.4 |
| 18 | RNase1 | 0.3 | 1.6 | 0.3 | 1.1 | 0.4 | 0.5 | 0.5 | 11.5 |
| 19 | DRP-2 | 0.9 | 8.9 | 6.4 | 2.4 | 5.9 | 5.7 | 7.9 | 1.1 |
| 22 | Iysosomal acid lipase | 3.4 | 4.2 | 0.3 | 8.1 | 0.3 | 0.4 | 0.5 | 6.1 |
| 23 | Cystatin A | 0.5 | 0.8 | 0.3 | 17.4 | 0.3 | 0.4 | 0.4 | 4.3 |
| 24 | CD36 | 0.4 | 0.8 | 0.8 | 2.9 | 0.8 | 0.8 | 0.9 | 1.2 |
| 26 | MCP1 | 0.3 | 0.7 | 0.2 | 0.5 | 0.2 | 0.3 | 3.3 | 9.6 |
| 28 | TGFBI | 1.8 | 1.8 | 0.5 | 3.4 | 1.5 | 0.9 | 0.7 | 9.2 |
| 30 | Calmodulin | 0.8 | 5.1 | 0.5 | 1.8 | 0.4 | 0.7 | 0.6 | 1.6 |
| 31 | MDL-1 | 0.4 | 1.0 | 0.4 | 1.3 | 0.6 | 0.8 | 0.7 | 1.9 |
| 33 | beta-tubulin | 1.1 | 2.8 | 0.4 | 2.8 | 0.3 | 0.3 | 0.6 | 2.7 |
| 37 | Enolase 1 | 0.6 | 3.8 | 0.3 | 1.4 | 0.1 | 0.1 | 0.4 | 1.5 |
| 40 | thymosin, beta 4 | 1.2 | 1.9 | 0.3 | 3.4 | 0.1 | 0.3 | 0.6 | 1.6 |
| 41 | Ferritin | 1.3 | 3.7 | 0.1 | 1.9 | 0.0 | 0.1 | 0.3 | 5.1 |
| 44 | Annexin A2 | 0.4 | 3.1 | 0.1 | 4.8 | 0.1 | 0.1 | 0.3 | 2.2 |
| 46 | MGST2 | 0.6 | 1.0 | 0.6 | 3.5 | 0.5 | 0.4 | 1.1 | 3.5 |
| 49 | factor XIII | 0.3 | 1.0 | 0.4 | 0.7 | 0.5 | 0.7 | 0.8 | 3.0 |
| 52 | VAMP8 | 2.0 | 2.8 | 0.6 | 5.1 | 0.3 | 0.2 | 1.3 | 4.2 |
| 54 | purinergic receptor | 0.4 | 1.0 | 0.6 | 3.1 | 0.6 | 0.6 | 1.0 | 1.9 |
| 55 | ARHGDIB | 0.1 | 0.2 | 0.1 | 1.0 | 0.1 | 0.1 | 0.3 | 2.0 |
| 58 | MHC DM-alpha | 0.8 | 1.9 | 1.3 | 4.1 | 0.6 | 0.4 | 2.2 | 2.8 |
| 61 | DORA | 1.7 | 0.9 | 0.5 | 4.0 | 0.4 | 0.6 | 0.7 | 1.8 |
| 63 | IRF4 | 0.3 | 7.9 | 10.0 | 2.2 | 9.3 | 0.9 | 5.8 | 0.4 |
| S2 | CCR1 | 0.3 | 0.8 | 0.3 | 0.8 | 0.3 | 0.5 | 0.4 | 1.6 |
| S3 | CCR7 | 0.1 | 3.1 | 0.3 | 1.5 | 0.1 | 0.1 | 0.1 | 0.1 |
| S4 | DC-Lamp | 0.3 | 7.4 | 1.4 | 6.3 | 0.4 | 0.8 | 0.5 | 0.4 |
| S5 | E-cadherin | 0.5 | 1.0 | 0.6 | 1.0 | 0.9 | 0.9 | 0.5 | 0.9 |
| S6 | DEC205 (CD205) | 0.4 | 2.9 | 0.3 | 1.6 | 0.8 | 0.6 | 0.5 | 0.7 |
| S7 | Man_receptor (CD206) | 1.1 | 1.8 | 3.1 | 6.6 | 0.4 | 0.5 | 2.0 | 8.6 |
| S8 | Langerin (CD207) | 0.4 | 1.2 | 0.5 | 5.9 | 0.8 | 0.7 | 0.6 | 1.0 |

Table 3 represents the analysis of DC-associated gene expressions in different maturation stages of each DC subset. Genes showing little difference between mature and immature stages were omitted from the list shown in Table 2.

Throughout this application, various patents and publications are referenced and citations are provided in parentheses. The disclosure of these patents and publications in their entities are hereby incorporated by referenced into this application in order to more fully describe this invention and the state of the art to which this invention pertains.

Having described a preferred embodiment of the present invention, it is to be understood that variants and modifications thereof falling within the spirit of the invention may become apparent to those skilled in this art, and the scope of this invention is to be determined by appended claims and their equivalents.

### REFERENCE

1. Hart D. N., Blood. 1997; 90: 3245-3287
2. Banchereau J. et al., Nature. 1998; 392: 245-252
3. Banchereau J. et al., Annu Rev Immunol. 2000; 18: 767-811
4. Ruedl C. et al., Eur J. Immunol. 2000; 30: 2056-2064
5. Pashenkov M. et al., Brain. 2001; 124: 480-492
6. Bernhard H. et al., Exp Hematol. 2000; 28: 365-372
7. Zhang Y. et al., Blood. 2000; 95: 138-146
8. Zhang Y. et al., Blood. 1999; 93: 1208-1220
9. Reid S. D. et al., Curr Opin Immunol. 2000; 12: 114-121
10. Grabbe S. et al., Immunol Today. 2000; 21: 431-433
11. Moser M. et al., Nat Immunol. 2000; 1: 199-205
12. Pulendran B. et al., Proc Natl Acad Sci USA. 1999; 96: 1036-1041
13. Steinman R. M. et al., J Exp Med. 2000; 191: 411-416
14. Vieira P. L. et al., J. Immunol. 2000; 164: 4507-4512
15. Tanaka H. et al., J Exp Med. 2000; 192: 405-412
16. Langenkamp A. et al., Nat Immunol. 2000; 1: 311-316
17. Patterson S. et al., Nat Immunol. 2000; 1: 273-274
18. Fong L. et al., J. Immunol. 1997; 159: 3113-3117
19. Fields R. C. et al., Proc Natl Acad Sci USA. 1998; 95: 9482-9487
20. Esche C. et al., Curr Opin Mol Ther. 1999; 1: 72-81
21. Fong L. et al., Annu Rev Immunol. 2000; 18: 245-273
22. Duperrier K. et al., J Immunol Methods. 2000; 238: 119-131
23. Dzionek A. et al., J. Immunol. 2000; 165: 6037-6046
24. Strunk D. et al., Blood. 1996; 87: 1292-1302
25. Caux C. et al., Blood. 1996; 87: 2376-2385
26. Hashimoto S. et al., Blood. 1999; 94: 845-852
27. Hashimoto S. I. et al., Blood. 2000; 96: 2206-2214
28. Dietz A. B. et al., Biochem Biophys Res Commun. 2000; 275: 731-738
29. Le Naour F. et al., J Biol. Chem. 2001; 276: 17920-17931
30. Kim K. et al., Mol. Cells. 1996; 6: 684-691
31. Bae Y. et al., Mol. Cells. 1995; 5: 569-578
32. Caux C. et al., Blood. 1997; 90: 1458-1470
33. de Saint-Vis B. et al., J. Immunol. 1998; 160: 1666-1676
34. McLellan A. D. et al., J Immunol Methods. 1995; 184: 81-89
35. Choi B. K. et al., Diabetes. 2000; 49: 1459-1467
36. Kim I. S. et al., J Biol. Chem. 2000; 275: 23139-23145
37. Valladeau J. et al., Eur J. Immunol. 1999; 29: 2695-2704
38. Diatchenko L. et al., Proc Natl Acad Sci USA. 1996; 93: 6025-6030
39. Mittrucker H. W. et al., Science. 1997; 275: 540-543
40. Bendriss-Vermare N. et al., J Clin Invest. 2001; 107: 835-844
41. Kadowaki N. et al., J Exp Med. 2001; 194: 863-869
42. Donaghy H. et al., Blood. 2001; 98: 2574-2576
43. Bjorck P. Blood. 2001; 98: 3520-3526
44. Funk C. D. Science. 2001; 294: 1871-1875
45. Vissers J. L. et al., J Leukoc Biol. 2001; 69: 785-793
46. Kadowaki N. et al., J Exp Med. 2000; 192: 219-226
47. Siegal F. P. et al., Science. 1999; 284: 1835-1837
48. Ito T. et al., J. Immunol. 2001; 166: 2961-2969
49. Sallusto F. et al., Eur J. Immunol. 1999; 29: 1617-1625
50. Ashkar S. et al., Science. 2000; 287: 860-864
51. Pacheco L. S. et al., J Cutan Pathol. 2000; 27: 136-140
52. Gibran N. S. et al., J Surg Res. 1995; 59: 378-386
53. Gibran N. S. et al., J Surg Res. 1997; 70: 1-6
54. de Saint-Vis B. et al., Immunity. 1998; 9: 325-336
55. Mohamadzadeh M. et al., J Exp Med. 2001; 194: 1013-1020
56. Borkowski T. A. et al., J Clin Invest. 1997; 100: 575-581
57. Geissmann F. et al., J Exp Med. 1998; 187: 961-966
58. Ito T. et al., J. Immunol. 1999; 163: 1409-1419
59. Jaksits S. et al., J. Immunol. 1999; 163: 4869-4877
60. Sato K. et al., J. Immunol. 2000; 164: 2285-2295
61. Skonier J. et al., DNA Cell Biol. 1994; 13: 571-584
62. Langnaese K. et al., Biochim Biophys Acta. 2000; 1492: 522-525
63. Weterman M. A. et al., Int J Cancer. 1995; 60: 73-81
64. Ishibashi K. et al., Gene. 2001; 264: 87-93
65. Bakker A. B. et al., Proc Natl Acad Sci USA. 1999; 96: 9792-9796
66. Oppel T. et al., Br J. Dermatol. 2000; 143: 1193-1198
67. Jahnsen F. L. et al., J. Immunol. 2000; 165: 4062-4068

1. A dendritic cell-specific polynucleotide comprising a nucleotide sequence of SEQ ID NOs:1, 3, 4, 5 or 6.

2. A dendritic cell-specific polypeptide encoded by a nucleotide sequence of SEQ ID NOs:1, 3, 4, 5 or 6.

3. A method for detecting a dendritic cell comprising the steps of:
(a) hybridizing a DNA obtained from a cell or its fragment with a dendritic cell-specific nucleotide sequence; and
(b) verifying the occurrence of the hybridization;
wherein said dendritic cell-specific nucleotide sequence is selected from the group consisting of myosin phosphatase target subunit 1 gene, CD20-like precursor gene, Ig superfamily protein gene, glycoprotein nmb gene, 5-lipoxygenase activating protein gene, dihydropyrimidinase related protein-2 gene, cystatin A gene, Immunoglobulin transcription factor 2 gene, transforming growth factor beta-induced 68 kD gene, myeloid DAP12-associating lectin gene, B cell linker protein gene, activated RNA polymerase II transcription cofactor 4 gene, enolase 1 alpha gene, 90 kDa heat shock protein gene, accessory proteins BAP31/BAP29 gene, isocitrate dehydrogenase 3 (NAD⁺) alpha gene, microsomal glutathione S-transferase 2 gene, GABA(A) receptor-associated protein gene, nicastrin gene, purinergic receptor (family A group 5) gene, Rho GDP dissociation inhibitor beta gene, MAD homolog 2 gene, MLN51 gene, interferon regulatory factor 4 gene, the fragments of these genes, a polynucleotide of SEQ ID NO:1 or its fragment, a polynucleotide of SEQ ID NO:2 or its fragment, a polynucleotide of SEQ ID NO:3 or its fragment, a polynucleotide of SEQ ID NO:4 or its fragment, a polynucleotide of SEQ ID NO:5 or its fragment, a polynucleotide of SEQ ID NO:6 or its fragment and the combination thereof.

4. A method for identifying a lymphoid CD11c⁻ dendritic cell comprising the steps of:
(a) hybridizing a DNA obtained from a cell or its fragment with a CD11c⁻ dendritic cell-specific nucleotide sequence; and
(b) verifying the occurrence of the hybridization;
wherein said lymphoid CD11c⁻ dendritic cell-specific nucleotide sequence is selected from the group consisting of 5-lipoxygenase activating protein gene or its fragment, dihydropyrimidinase related protein-2 gene or its fragment, interferon regulatory factor 4 gene or its fragment and the combination thereof.

5. A method for identifying a myeloid monocyte-derived dendritic cell comprising the steps of:
(a) hybridizing a DNA obtained from a cell or its fragment with a myeloid monocyte-derived dendritic cell-specific nucleotide sequence; and
(b) verifying the occurrence of the hybridization;
wherein said myeloid monocyte-derived dendritic cell-specific nucleotide sequence is selected from the group consisting of thymus and activation-regulated chemokine gene or its fragment, dihydropyrimidinase related protein-2 gene or its fragment, lysosomal acid lipase gene or its fragment, calmodulin gene or its fragment, interferon regulatory factor 4 gene or its fragment, DC-Lamp gene or its fragment and the combination thereof.

6. A method for identifying a myeloid CD1a⁺ dendritic cell comprising the steps of:
(a) hybridizing a DNA obtained from a cell or its fragment with a myeloid CD1a⁺ dendritic cell-specific nucleotide sequence; and
(b) verifying the occurrence of the hybridization;
wherein said myeloid CD1a⁺ dendritic cell-specific nucleotide sequence is selected from the group consisting of a polynucleotide of SEQ ID NO:2 or its fragment, a polynucleotide of SEQ ID NO:3 or its fragment, a polynucleotide of SEQ ID NO:5 or its fragment, S100 calcium-binding protein beta gene or its fragment, matrix metalloproteinase 12 gene or its fragment, thymus and activation-regulated chemokine gene or its fragment, CD1B antigen gene or its fragment, CD20-like precursor gene or its fragment, MHC class II HLA-DQ-alpha chain gene or its fragment, osteopontin gene or its fragment, 5-lipoxygenase activating protein gene or its fragment, monocyte chemotactic proteins 4 gene or its fragment, lysosomal acid lipase gene or its fragment, cystatin A gene or its fragment, annexin A2 gene or its fragment, vesicle-associated membrane protein 8 gene or its fragment, MHC class II HLA-DM-alpha chain gene or its fragment, DORA protein gene or its fragment, DC-Lamp gene or its fragment, Mannose receptor (CD206) gene or its fragment, Langerin (CD207) gene or its fragment and the combination thereof.

7. A method for identifying a myeloid CD14⁺ dendritic cell comprising the steps of:
(a) hybridizing a DNA obtained from a cell or its fragment with a myeloid CD14⁺ dendritic cell-specific nucleotide sequence; and
(b) verifying the occurrence of the hybridization;
wherein said myeloid CD14⁺ dendritic cell-specific nucleotide sequence is selected from the group consisting of a polynucleotide of SEQ ID NO:2 or its fragment, S100 calcium-binding protein beta gene or its fragment, myosin phosphatase target subunit 1 gene or its fragment, CD20-like precursor gene or its fragment, Ig superfamily protein gene or its fragment, glycoprotein nmb gene or its fragment, osteopontin gene or its fragment, 5-lipoxygenase activating protein gene or its fragment, mannose receptor C type 1 gene or its fragment, monocyte chemotactic proteins 4 gene or its fragment, RNase A family 1 (RNas1) gene or its fragment, lysosomal acid lipase gene or its fragment, cystatin A gene or its fragment, monocyte chemotactic proteins 1 (MCP 1) gene or its fragment, transforming growth factor beta-induced 68 kD gene or its fragment, ferritin light polypeptide gene or its fragment, vesicle-associated membrane protein 8 gene or its fragment, Mannose receptor (CD206) gene or its fragment and the combination thereof.

8. A method for identifying a maturation stage of a lymphoid CD11c⁻ dendritic cell comprising the steps of:
(a) hybridizing a DNA obtained from a cell or its fragment with an interferon regulatory factor 4 gene or its fragment; and
(b) verifying the occurrence of the hybridization.

9. A method for identifying a maturation stage of a myeloid monocyte-derived dendritic cell comprising the steps of:
(a) hybridizing a DNA obtained from a cell or its fragment with a nucleotide sequence; and
(b) verifying the occurrence of the hybridization;
wherein said nucleotide sequence is selected from the group consisting of thymus and activation-regulated chemokine gene or its fragment, dihydropyrimidinase related protein-2 gene or its fragment, interferon regulatory factor 4 gene or its fragment, DC-Lamp gene or its fragment and the combination thereof.

10. A method for identifying a maturation stage of a myeloid CD1a⁺ dendritic cell comprising the steps of:
(a) hybridizing a DNA obtained from a cell or its fragment with a nucleotide sequence; and
(b) verifying the occurrence of the hybridization;
wherein said nucleotide sequence is selected from the group consisting of a polynucleotide of SEQ ID NO:2 or its fragment, a polynucleotide of SEQ ID NO:3 or its fragment, a polynucleotide of SEQ ID NO:5 or its fragment, S100 calcium-binding protein beta gene or its fragment, matrix metalloproteinase 12 gene or its fragment, thymus and activation-regulated chemokine gene or its fragment, CD1B antigen gene or its fragment, CD20-like precursor gene or its fragment, MHC class II HLA-DQ-alpha chain gene or its fragment, osteopontin gene or its fragment, monocyte chemotactic proteins 4 gene or its fragment, lysosomal acid lipase gene or its fragment, cystatin A gene or its fragment, transforming growth factor beta-induced 68 kD gene or its fragment, annexin A2 gene or its fragment, vesicle-associated membrane protein 8 gene or its fragment, DORA protein gene or its fragment, DC-Lamp gene or its fragment, Langerin (CD207) gene or its fragment and the combination thereof.

11. A method for identifying a maturation stage of a myeloid CD14⁺ dendritic cell comprising the steps of:
(a) hybridizing a DNA obtained from a cell or its fragment with a nucleotide sequence; and
(b) verifying the occurrence of the hybridization;
wherein said nucleotide sequence is selected from the group consisting of a polynucleotide of SEQ ID NO:2 or its fragment, S100 calcium-binding protein beta gene or its fragment, CD20-like precursor gene or its fragment, Ig superfamily protein gene or its fragment, glycoprotein nmb gene or its fragment, osteopontin gene or its fragment, 5-lipoxygenase activating protein gene or its fragment, mannose receptor C type 1 gene or its fragment, monocyte chemotactic proteins 4 gene or its fragment, RNase A family 1 gene or its fragment, lysosomal acid lipase gene or its fragment, cystatin A gene or its fragment, monocyte chemotactic proteins 1 gene or its fragment, transforming growth factor beta-induced 68 kD gene or its fragment, ferritin light polypeptide gene or its fragment, vesicle-associated membrane protein 8 gene or its fragment, Mannose receptor (CD206) gene or its fragment and the combination thereof.

12. A microarray for detecting a dendritic cell comprising a dendritic cell-specific nucleotide sequence immobilized on a solid surface;
wherein said dendritic cell-specific nucleotide sequence is selected from the group consisting of myosin phosphatase target subunit 1 gene, CD20-like precursor gene, Ig superfamily protein gene, glycoprotein nmb gene, 5-lipoxygenase activating protein gene, dihydropyrimidinase related protein-2 gene, cystatin A gene, Immunoglobulin transcription factor 2 gene, transforming growth factor beta-induced 68 kD gene, myeloid DAP12-associating lectin gene, B cell linker protein gene, activated RNA polymerase II transcription cofactor 4 gene, enolase 1 alpha gene, 90 kDa heat shock protein gene, accessory proteins BAP31/BAP29 gene, isocitrate dehydrogenase 3 (NAD⁺) alpha gene, microsomal glutathione S-transferase 2 gene, GABA(A) receptor-associated protein gene, nicastrin gene, purinergic receptor (family A group 5) gene, Rho GDP dissociation inhibitor beta gene, MAD homolog 2 gene, MLN51 gene, interferon regulatory factor 4 gene, the fragments of these genes, a polynucleotide of SEQ ID NO:1 or its fragment, a polynucleotide of SEQ ID NO:2 or its fragment, a polynucleotide of SEQ ID NO:3 or its fragment, a polynucleotide of SEQ ID NO:4 or its fragment, a polynucleotide of SEQ ID NO:5 or its fragment, a polynucleotide of SEQ ID NO:6 or its fragment and the combination thereof.

13. A microarray for identifying a lymphoid CD11c⁻ dendritic cell comprising a lymphoid CD11c⁻ dendritic cell-specific nucleotide sequence immobilized on a solid surface;
wherein said lymphoid CD11c⁻ dendritic cell-specific nucleotide sequence is selected from the group consisting of 5-lipoxygenase activating protein gene or its fragment, dihydropyrimidinase related protein-2 gene or its fragment, interferon regulatory factor 4 gene or its fragment and the combination thereof.

14. A microarray for identifying a myeloid monocyte-derived dendritic cell comprising a myeloid monocyte-derived dendritic cell-specific nucleotide sequence immobilized on a solid surface;
wherein said myeloid monocyte-derived dendritic cell-specific nucleotide sequence is selected from the group consisting of thymus and activation-regulated chemokine gene or its fragment, dihydropyrimidinase related protein-2 gene or its fragment, lysosomal acid lipase or its fragment, calmodulin gene or its fragment, interferon regulatory factor 4 gene or its fragment, DC-Lamp gene or its fragment and the combination thereof.

15. A microarray for identifying a myeloid CD1a⁺ dendritic cell comprising a myeloid CD1a⁺ dendritic cell-specific nucleotide sequence immobilized on a solid surface;
wherein said myeloid CD1a⁺ dendritic cell-specific nucleotide sequence is selected from the group consisting of a polynucleotide of SEQ ID NO:2 or its fragment, a polynucleotide of SEQ ID NO:3 or its fragment, a polynucleotide of SEQ ID NO:5 or its fragment, S100 calcium-binding protein beta gene or its fragment, matrix metalloproteinase 12 gene or its fragment, thymus and activation-regulated chemokine gene or its fragment, CD1B antigen gene or its fragment, CD20-like precursor gene or its fragment, MHC class II HLA-DQ-alpha chain gene or its fragment, osteopontin gene or its fragment, 5-lipoxygenase activating protein gene or its fragment, monocyte chemotactic proteins 4 gene or its fragment, lysosomal acid lipase gene or its fragment, cystatin A gene or its fragment, annexin A2 gene or its fragment, vesicle-associated membrane protein 8 gene or its fragment, MHC class II HLA-DM-alpha chain gene or its fragment, DORA protein gene or its fragment, DC-Lamp gene or its fragment, Mannose receptor (CD206) gene or its fragment, Langerin (CD207) gene or its fragment and the combination thereof.

16. A microarray for identifying a myeloid CD14⁺ dendritic cell comprising a myeloid CD14⁺ dendritic cell-specific nucleotide sequence immobilized on a solid surface;
wherein said myeloid CD14⁺ dendritic cell-specific nucleotide sequence is selected from the group consisting of a polynucleotide of SEQ ID NO:2 or its fragment, S100 calcium-binding protein beta gene or its fragment, myosin phosphatase target subunit 1 gene or its fragment, CD20-like precursor gene or its fragment, Ig superfamily protein gene or its fragment, glycoprotein nmb gene or its fragment, osteopontin gene or its fragment, 5-lipoxygenase activating protein gene or its fragment, mannose receptor C type 1 gene or its fragment, monocyte chemotactic proteins 4 gene or its fragment, RNase A family 1 (RNas 1) gene or its fragment, lysosomal acid lipase gene or its fragment, cystatin A gene or its fragment, monocyte chemotactic proteins 1 (MCP 1) gene or its fragment, transforming growth factor beta-induced 68 kD gene or its fragment, ferritin light polypeptide gene or its fragment, vesicle-associated membrane protein 8 gene or its fragment, Mannose receptor (CD206) gene or its fragment and the combination thereof.

17. A microarray for identifying a maturation stage of a lymphoid CD11c⁻ dendritic cell comprising an interferon regulatory factor 4 gene or its fragment immobilized on a solid surface.

18. A microarray for identifying a maturation stage of a myeloid monocyte-derived dendritic cell comprising a nucleotide sequence immobilized on a solid surface;
wherein said nucleotide sequence is selected from the group consisting of thymus and activation-regulated chemokine gene or its fragment, dihydropyrimidinase related protein-2 gene or its fragment, interferon regulatory factor 4 gene or its fragment, DC-Lamp gene or its fragment and the combination thereof.

19. A microarray for identifying a maturation stage of a myeloid CD1a⁺ dendritic cell comprising a nucleotide sequence immobilized on a solid surface;
wherein said nucleotide sequence is selected from the group consisting of a polynucleotide of SEQ ID NO:2 or its fragment, a polynucleotide of SEQ ID NO:3 or its fragment, a polynucleotide of SEQ ID NO:5 or its fragment, S100 calcium-binding protein beta gene or its fragment, matrix metalloproteinase 12 gene or its fragment, thymus and activation-regulated chemokine gene or its fragment, CD1B antigen gene or its fragment, CD20-like precursor gene or its fragment, MHC class II HLA-DQ-alpha chain gene or its fragment, osteopontin gene or its fragment, monocyte chemotactic proteins 4 gene or its fragment, lysosomal acid lipase gene or its fragment, cystatin A gene or its fragment, transforming growth factor beta-induced 68 kD gene or its fragment, annexin A2 gene or its fragment, vesicle-associated membrane protein 8 gene or its fragment, DORA protein gene or its fragment, DC-Lamp gene or its fragment, Langerin (CD207) gene or its fragment and the combination thereof.

20. A microarray for identifying a maturation stage of a myeloid CD14⁺ dendritic cell comprising a nucleotide sequence immobilized on a solid surface;
wherein said nucleotide sequence is selected from the group consisting of a polynucleotide of SEQ ID NO:2 or its fragment, S100 calcium-binding protein beta gene or its fragment, CD20-like precursor gene or its fragment, Ig superfamily protein gene or its fragment, glycoprotein nmb gene or its fragment, osteopontin gene or its fragment, 5-lipoxygenase activating protein gene or its fragment, mannose receptor C type 1 gene or its fragment, monocyte chemotactic proteins 4 gene or its fragment, RNase A family 1 gene or its fragment, lysosomal acid lipase gene or its fragment, cystatin A gene or its fragment, monocyte chemotactic proteins 1 gene or its fragment, transforming growth factor beta-induced 68 kD gene or its fragment, ferritin light polypeptide gene or its fragment, vesicle-associated membrane protein 8 gene or its fragment, Mannose receptor (CD206) gene or its fragment and the combination thereof.
